SEQUENCE LISTING (16 sequences)

SEQ ID NO: 1 - DNA, 19 bp, Artificial Sequence
  Synthetic oligonucleotide
gcgccattcaccgcggcgc

SEQ ID NO: 2 - DNA, 25 bp, Artificial Sequence
  Synthetic oligonucleotide
gcgccattcaccgcgtgcctggcgc

SEQ ID NO: 3 - DNA, 31 bp, Artificial Sequence
  Synthetic oligonucleotide
gcgccatcattcaccgcgtgccttttggcgc

SEQ ID NO: 4 - DNA, 37 bp, Artificial Sequence
  Synthetic oligonucleotide
gcgccattttcattcaccgcgtgcctttttttggcgc

SEQ ID NO: 5 - DNA, 43 bp, Artificial Sequence
  Synthetic oligonucleotide
gcgccatttttttcattcaccgcgtgccttttttttttggcgc

SEQ ID NO: 6 - DNA, 49 bp, Artificial Sequence
  Synthetic oligonucleotide
gcgccattttttttttcattcaccgcgtgcctttttttttttttggcgc

SEQ ID NO: 7 - DNA, 25 bp, Artificial Sequence
  Synthetic oligonulceotide
gcgccagctaacaatacactggcgc

SEQ ID NO: 8 - DNA, 22 bp, Artificial Sequence
  Synthetic oligonucleotide
ttaaggcacgcggtgaatgcca

SEQ ID NO: 9 - DNA, 22 bp, Artificial Sequence
  Synthetic oligonucleotide
ttaaggcacgtggtgaatgcca

SEQ ID NO: 10 - DNA, 24 bp, Artificial Sequence
  Synthetic oligonucleotide
tcacaacctcctagaaagagtaga

SEQ ID NO: 11 - DNA, 20 bp, Artificial Sequence
  Synthetic oligonucleotide
ccgcatcttcttttgcgtcg

SEQ ID NO: 12 - DNA, 21 bp, Artificial Sequence
  Synthetic oligonucleotide
gcccaatacgaccaaatccgt

SEQ ID NO: 13 - DNA, 19 bp, Artificial Sequence
  Synthetic oligonucleotide
caacagcgacggaggtctc

SEQ ID NO: 14 - DNA, 22 bp, Artificial Sequence
  Synthetic oligonucleotide
gcctctacgctctcttctttga

SEQ ID NO: 15 - DNA, 21 bp, Artificial Sequence
  Synthetic oligonucleotide
ggaccctgtgagtagccagta

SEQ ID NO: 16 - DNA, 19 bp, Artificial Sequence
  Synthetic oligonucleotide
cagctccgacagatccagt

(19) United States
(12) Patent Application Publication
(10) Pub. No.: US 2021/0215679 A1
(43) Pub. Date: Jul. 15, 2021

(54) MAGNETO-PLASMONIC NANOMATERIALS AND METHODS OF USE

(71) Applicants: Rutgers, The State University of New Jersey, New Brunswick, NJ (US); Sogang University, Seoul (KR)

(72) Inventors: Ki-Bum Lee, Piscataway, NJ (US); Jin-Ho Lee, Piscataway, NJ (US); Jeong-Woo Choi, Seoul (KR); Jin-Ha Choi, Seoul (KR)

(21) Appl. No.: 17/142,950

(22) Filed: Jan. 6, 2021

Related U.S. Application Data
(60) Provisional application No. 62/958,166, filed on Jan. 7, 2020.

(51) Int. Cl. G01N 33/533; H01F 1/40; H01F 3/06; G01N 21/64

(52) U.S. Cl. CPC G01N 33/533; H01F 1/40; B82Y 25/00; G01N 21/6428; H01F 3/06

(57) ABSTRACT

Nanorod devices for isolating and characterizing target cellular components are provided. Methods of isolating, detecting, and/or characterizing the components are also provided. Methods of use and treatment are further disclosed, such as treating diseases identified using the nanorods and/or using differentiated stem cells identified using the provided nanorods.

### CROSS REFERENCE TO RELATED APPLICATION

This application claims the benefit of U.S. Provisional Application No. 62/958,166, filed Jan. 7, 2020, which is incorporated herein by reference in its entirety.

### ACKNOWLEDGMENT OF GOVERNMENT SUPPORT

This invention was made with government support under grant numbers 1R21NS085569/R21AR071101 and 1R01DC016612-01/3R01DC016612-01S1/3R01DC016612-02S1 awarded by the National Institutes of Health and CHE-1429062/BCET-1803517 awarded by the National Science Foundation. The government has certain rights in the invention.

This invention was also made with support under National Research Foundation of Korea (NRF) grant No.2019R1A2C3002300, funded by the Ministry of Science and ICT of South Korea.

### FIELD

This application provides magneto-plasmonic nanorods and methods of use, particularly for isolating, detecting, and/or characterizing extracellular vesicles.

### BACKGROUND

Stem cells hold great potential for the treatment of neurodegenerative diseases and central nervous system (CNS) injuries (Ascherio et al., Lancet Neurol. 15:1257-1272, 2016; Logroscino et al., Lancet Neurol. 17:1083-1097, 2018; Trounson et al., Nat. Rev. Mol. Cell Biol. 17:194-200, 2016; Fox et al., Science 345:1247391, 2014). However, precise control and characterization of stem cell fate are important for therapeutic applications (Lee et al., Nat. Med. 19:998, 2013; Shi et al., Nat. Rev. Drug Discovery 16:115, 2017; Chen et al., Cell Stem Cell 14:13-26, 2014; Dimmeler et al., Nat. Med. 20:814-821, 2014). Previous characterization methods for stem cell biomarkers have been intrinsically destructive to cellular activities and functions, which limits further analyses and biomedical applications (Goodwin et al., Biol. Blood Marrow Transplant. 7:581-588, 2001; Lee et al., Adv. Mater. 30:1802762, 2018).

Further, cell-cell interactions and intrinsic cellular regulators control stem cell neurogenesis through noncoding RNAs (e.g., microRNAs (miRNAs); Zhang et al., eLife 5:e11324, 2016; Yao et al., Nat. Rev. Neurosci. 17:537-549, 2016). For example, miRNAs in stem cell-derived exosomes regulate stem cell neurogenesis. Exosomes are actively secreted by mammalian cells (including stem cells) and are cellular components for intercellular communication (Ratajczak et al., Leukemia 20:847-856, 2006; Vlassov et al., Biochim. Biophys. Acta, Gen. Subj. 1820:940-948, 2012; Thery et al., Nat. Rev. Immunol. 9:581-593, 2009; Im et al., Nat. Biotechnol. 32:490-495, 2014). These membrane-bound phospholipid nanovesicles (approximately 50-100 nm in diameter) are physically stable and include signaling molecules (such as proteins, RNAs, and DNAs) to facilitate cell-cell communication (Shao et al., Chem. Rev. 118:1917-1950, 2018). Small noncoding RNAs (miRNAs; approximately 76% of total oligonucleotides inside of exosomes) can act as post-transcriptional gene regulators, which are involved in the determination of cell fate (Huang et al., BMC Genomics 14:319, 2013; Lund et al., Science 303:95-98, 2004; Lai, E. C., Nat. Genet. 30:363, 2002). For example, exosomal miRNA secreted from donor cells can target acceptor cells and directly modulate gene expression (Valadi et al., Nat. Cell Biol. 9:654-659, 2007; van Niel et al., Nat. Rev. Mol. Cell Biol. 19:213-228, 2018; Tkach et al., Cell 164:1226-1232, 2016; Pegtel et al., Proc. Natl. Acad. Sci. U.S.A. 107:6328-6333, 2010). Previous exosome analysis procedures require large sample volumes and specialized tools (such as an ultrahigh centrifuge with 100,000×g and a specific filtration membrane) to compensate for weak signals and poor isolation efficiency (Shao et al., Chem. Rev. 118:1917-1950, 2018; Greening et al., In Proteomic Profiling, Springer, pp 179-209, 2015; Melo et al., Nature 523:177-182, 2015; Taylor et al., In Serum/Plasma Proteomics, Springer, pp 235-246, 2011).

### SUMMARY

Improved methods of purifying, detecting, and characterizing target molecules, such as nucleic acids or extracellular vesicles (EVs), for example, exosomes, are needed. Furthermore, detection methods that do not compromise cellular viability, such as an accurate and simple analysis of exosomal molecules (especially miRNAs) from each cell stage, are desirable, for example, an isolation and detection approach for exosomal miRNA in a nondestructive, selective, and sensitive manner. Therefore, disclosed herein are devices for isolating, detecting, and characterizing cellular components (for example, EVs), as well as methods of use.

In some embodiments, disclosed herein are magneto-plasmonic nanorods for detecting and characterizing vesicles. In example embodiments, the nanorods include a cylindrical body with a center portion including a magnetic composition, flanked by end portions including a plasmonic composition. The magnetic composition is coupled to at least one immunoactive macromolecule (such as an antibody or an aptamer). In some examples, the plasmonic composition is coupled to a detectable molecule, for example a peptide or nucleic acid (such as a molecular beacon).

In example embodiments, the magnetic composition is nickel, iron, or cobalt. In specific, non-limiting examples, the magnetic composition is nickel. In example embodiments, the immunoactive macromolecule coupled to the magnetic composition (such as an antibody) specifically binds a specific type of vesicle (such as an exosome). In one example, the immunoactive macromolecule specifically binds to CD63.

In example embodiments, the detectable molecule is a labeled peptide or a labeled nucleic acid. In specific, non-limiting examples, the detectable molecule is a labeled nucleic acid (such as a labeled single-strand hairpin DNA, for example, a molecular beacon). In some examples, the detectable molecule is a labeled nucleic acid that specifically binds at least one target nucleic acid associated with a specific vesicle (such as an miRNA, for example, miR-124 or miR-449a).

In specific, non-limiting examples, the devices include nanorods with a cylindrical body including a center portion including a magnetic composition coupled to at least one antibody that specifically binds a specific vesicle or cell type and flanked by a plasmonic composition on each end which is coupled to a fluorescently labeled single strand hairpin DNA. Any one of the devices disclosed herein can also be included in a kit with instructions for use.

Further disclosed herein are methods of isolating a target cellular component, such as an exosome or nucleic acid, from a sample (such as a biological sample from a subject or culture media from a population of cells or tissue). In example embodiments, the methods include contacting a sample with the a nanorod disclosed herein under conditions sufficient for the nanorod to specifically bind to the target cellular component, isolating the nanorod bound to the target cellular component from the sample, and purifying the target cellular component from the nanorod.

Also disclosed herein are methods of detecting a target nucleic acid. In example embodiments, the methods include contacting a sample (such as a biological sample from a subject or culture media from a population of cells or tissue) with a nanorod disclosed herein under conditions sufficient for the nanorod to specifically bind to the target nucleic acid, isolating the nanorod bound to the target nucleic acid from the sample, measuring a signal from the isolated nanorod bound to the target nucleic acid (such as a fluorescent signal), and detecting the nucleic acid based on the signal.

Methods of treating a disease or disorder in a subject are also disclosed herein. In example embodiments, the methods include contacting any of the nanorods discloses herein with a biological sample from a subject or culture media from a population of cells or tissue from the subject under conditions sufficient for the nanorod to bind to a target molecule, isolating the nanorod bound to the target molecule, measuring a signal from the nanorod bound to the target molecule (such as a fluorescent signal), identifying the disease or disorder based on the signal, and administering a treatment for the disease or disorder to the subject. In some examples, the treatment is administering stem cells to the subject.

In some examples of the disclosed methods, the fluorescent signal is from a fluorophore coupled to a labeled nucleic acid coupled to the plasmonic material, and measuring the signal from the nanorod includes applying an excitation wavelength and detecting an emission wavelength. In some examples of the disclosed methods, isolating the nanorod includes contacting the device with a magnetic field source.

The foregoing and other features of the disclosure will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C show schematic diagrams illustrating exemplary methods for nondestructive, selective, and sensitive characterization of stem cell differentiation through exosomal miRNA detection. Multifunctional magneto-plasmonic nanorods are utilized to extract and concentrate exosomes immunomagnetically (FIG. 1A), analyze exosomal miRNA sensitively via metal-enhanced fluorescence effect (FIG. 1B), and/or monitor stem cell differentiation nondestructively (FIG. 1C).

FIGS. 2A-2H show generation and characterization of multicomponent magneto-plasmonic nanorods (NRs). FIG. 2A is a schematic illustration of an exemplary method to generate multicomponent magneto-plasmonic NRs using an electrochemical deposition method on an anodized aluminum oxide template. FIG. 2B shows an optical image of the magnetophoretic separation property of multicomponent magneto-plasmonic NRs and FIG. 2C shows corresponding UV-vis-NIR absorption spectrum of multicomponent magneto-plasmonic NRs. FIG. 2D is a representative SEM image of multicomponent magneto-plasmonic NRs (scale bar: 200 nm). FIG. 2E shows energy-dispersive X-ray spectroscopy spectrum (EDS) of as-prepared multicomponent magneto-plasmonic NRs. FIG. 2F is representative SEM images of single multicomponent magneto-plasmonic NR (left) and corresponding EDS element mappings (right) (scale bar: 100 nm). FIG. 2G is a graph showing EDS elemental line profiles of multicomponent magneto-plasmonic NRs. FIG. 2H is a graph showing size distributions (diameter and length) of as-synthesized multicomponent magneto-plasmonic NRs.

FIGS. 3A-3E show surface functionalization of multicomponent magneto-plasmonic nanorods for miRNA detection. FIG. 3A shows side (middle) and top views (right and left) of EM field intensity (E-E₀) of multicomponent magneto-plasmonic NRs under three-dimensional finite-difference time-domain simulation using 490 nm wavelength light. FIG. 3B is a schematic illustration of fluorescence intensities from quenched, metal-enhanced fluorescence, and free fluorescence with respect to the folded, unfolded, and cleaved MB from the Au surface of magneto-plasmonic NRs. FIG. 3C is a schematic illustration and calculated fluorescence signal intensities based on different lengths (bp) of molecular beacons. FIG. 3D is a graph showing fluorescence signal value obtained from the positive complementary miR-124 DNA sequence, single mismatched synthetic DNA sequence, and mature miRNA67 DNA sequence (negative target) using a solution assay. FIG. 3E shows linear correlations between concentrations (range from 1 pM to 1 μM) of miRNA-124 and observed fluorescence signals. The error bars represent mean±SD; n=3, **p<0.01 by one-way ANOVA with Tukey's post hoc test.

FIGS. 4A-4G show nondestructive, selective, and sensitive characterization of neurogenesis of hiPSC-NSCs through immunomagnetically concentrated exosomal miRNA detection. FIG. 4A is a schematic illustration of an exemplary method of nondestructive, selective, and sensitive characterization of stem cell differentiation through immunomagnetically concentrated exosomes using multifunctional magneto-plasmonic NRs. FIG. 4B shows representative immunocytochemistry images of hiPSC-NSC differentiation into neurons. Nucleus (Hoechst, blue), Nestin (Alexa Fluor 488, green), and TuJ1 (Alexa Fluor 647, red) (scale bar=100 μm). FIG. 4C is a graph of PCR analyses of cell lysate for a neuronal marker, TuJ1, and miRNA-124 expression during the neuronal differentiation period (range from D1 to D22). FIG. 4D is a representative scanning electron microscope (SEM) image of captured exosomes on the surface of multifunctional magneto-plasmonic NRs (scale bar=100 μm). FIG. 4E shows absorbance intensities obtained from HRP-TMB reaction (630 nm) in the absence and presence of anti-CD63 antibody on the surface of magneto-plasmonic NRs. FIG. 4F is a graph of fluorescence signals from time-dependent monitoring (range from D1 to D22) of hiPSC-NSCs during neuronal differentiation. A total of 3.0×10⁶ cells were seeded on a 9.5 cm² surface area and treated with fresh hiPSC-NSC media without growth factors. FIG. 4G is a graph of fluorescence signals for other cell types from each lineage (ectoderm, neuron, hiPSC-NSC, and astrocyte; mesoderm, mesenchymal stem cell; endoderm, vascular endothelial, and dermal fibroblast). The error bars represent mean±SD; n=3, **p<0.01 by one-way ANOVA with Tukey's post hoc test.

FIGS. 5A-5D show nondestructive characterization of neural cell distribution in explanted brain tissues. FIG. 5A is a schematic illustration of an exemplary method for nondestructive, selective, and sensitive characterization of dissected brain tissue explants using multifunctional magneto-plasmonic NRs. FIGS. 5B-5C are graphs of relative fluorescence signal values from neurons (FIG. 5B) and astrocytes (FIG. 5C). FIG. 5D shows representative immunocytochemistry staining images of brain tissue explants and relative fluorescence signals obtained from multifunctional magneto-plasmonic NRs for each miRNA expression from the different cross-sections of brain tissue explants. Nucleus (Hoechst, blue), GFAP (Alexa Fluor 488, green), and TuJ1 (Alexa Fluor 647, red) (scale bar=100 μm). The error bars represent mean±SD; n=3, **p<0.01 by one-way ANOVA with Tukey's post hoc test.

FIGS. 6A and 6B show the correlation between coulomb value applied for electrochemical growth of Au (FIG. 6A) and Ni (FIG. 6B) using anodized aluminum oxide (AAO, ANODISC™ 13, pore size 0.2 μm, WHATMAN™) as a template.

FIG. 7 shows excitation and emission bands of a fluorescent dye (6-carboxyfluorescein, FAM) and absorbance band of magneto-plasmonic NRs.

FIGS. 8A-8C show relative fluorescence signal values obtained by multifunctional magneto-plasmonic NRs from neuron populations (FIG. 8A), astrocyte populations (FIG. 8B), and neuron/astrocytes co-cultured with different ratios (total number of cells ˜2.0×10⁶) (FIG. 8C).

FIGS. 9A-9C show a comparison of exosome isolation efficiency and specificity between the ultracentrifugation method and immunomagnetic isolation method. FIG. 9A shows schematic diagram of ultracentrifugation (UC) and immunomagnetic (IM) isolation methods. FIGS. 9B and 9C show a comparison of exosome isolation efficiency by the two methods using lipid quantification (FIG. 9B) and protein quantification (BCA assay) (FIG. 9C).

### SEQUENCE LISTING

Any nucleic acid and amino acid sequences listed herein or in the accompanying sequence listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

The Sequence Listing is submitted as an ASCII text file in the form of the file named Sequence_Listing.txt, which was created on Jan. 6, 2021, and is ˜4 kilobytes, which is incorporated by reference herein.

SEQ ID NOs: 1-7 are exemplary molecular beacon sequences.

SEQ ID NOs: 8-10 are exemplary control sequences.

SEQ ID NOs: 11-16 are exemplary primer sequences for analyzed genes.

### DETAILED DESCRIPTION

### I. Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a cell” includes single or plural subjects and is considered equivalent to the phrase “comprising at least one subject.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, patent applications and publications are incorporated by reference in their entirety. GENBANK® Accession numbers cited herein are incorporated by reference as present in the database on Jan. 7, 2020. Unless otherwise indicated, “about” indicates within five percent.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Administration: The introduction of a composition (such as stem cells or a pharmaceutical preparation that includes stem cells) into a subject by a chosen route. The route can be local or systemic. For example, if the chosen route is intravenous, the composition is administered by introducing the composition into a vein of the subject. If the chosen route is local, the composition can be administered by introducing the composition directly into a tissue of the subject.

Aptamer: Oligonucleotide or peptide molecules that bind to a specific target molecule. In some examples, aptamers are short, single-stranded DNA or RNA (ssDNA or ssRNA) molecules that can selectively bind to a specific target, including proteins, peptides, carbohydrates, small molecules, toxins, and even live cells. Aptamers assume a variety of shapes due to their tendency to form helices and single-stranded loops (such as a hairpin). In specific examples, an aptamer is coupled with a magnetic portion of a nanorod (such as a nanorod disclosed herein). In specific examples, an aptamer is coupled with a plasmonic portion of a nanorod (such as disclosed herein).

Cell Culture: Cells grown under controlled or defined conditions. A primary cell culture is a culture of cells, tissues, or organs taken directly from an organism. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Complementary: A nucleic acid molecule is said to be complementary to another nucleic acid molecule if the two molecules share a sufficient number of complementary nucleotides (for example, A-T, A-U, or G-C) to form a stable duplex or triplex when the strands bind (hybridize) to each other, for example by forming Watson-Crick, Hoogsteen, or reverse Hoogsteen base pairs. Stable or specific binding occurs when a nucleic acid molecule (e.g., a single stranded DNA, such as a single stranded DNA molecule that forms a hairpin) remains detectably bound to another nucleic acid (e.g., a target nucleic acid, such as a target miRNA) as a result of base pairing between complementary nucleotides in the nucleic acid molecules under the required conditions.

Complementarity is the degree to which bases in one nucleic acid molecule (e.g., a labeled nucleic acid) base pair with the bases in a second nucleic acid molecule (e.g., target nucleic acid). Complementarity is conveniently described by percentage, that is, the proportion of nucleotides that form base pairs between two molecules or within a specific region or domain of two molecules. For example, if 10 nucleotides of a 15 contiguous nucleotide region of a labeled nucleic acid form base pairs with a target nucleic acid, that region of the labeled nucleic acid is said to have 66.67% complementarity to the target nucleic acid. In some examples herein, two nucleic acids (such as a target nucleic acid and a labeled nucleic acid) are 100% complementary, while in other examples, two nucleic acid molecules are at least 80% complementary (for example, at least 85%, at least 90%, at least 95%, at least 98%, or more complementary).

Detect: To determine if an agent (such as a vesicle or a target nucleic acid) is present or absent. In some examples, this can further include quantification. For example, use of the disclosed methods and labels in particular examples permits determination of presence, amount, and/or identity of a vesicle, such as an exosome, and/or a target nucleic acid in a sample.

Detectable label: A compound or composition that is conjugated directly or indirectly to another molecule (such as an aptamer or molecular beacon, for example, a nucleic acid) to facilitate detection of that molecule, producing a detectable molecule. Specific, non-limiting examples of detectable labels include fluorescent and fluorogenic moieties, chromogenic moieties, haptens, affinity tags, and radioactive isotopes. The label can be directly detectable (e.g., optically detectable) or indirectly detectable (for example, via interaction with one or more additional molecules that are in turn detectable). Exemplary labels in the context of the detection probes disclosed herein are described below. Methods for labeling nucleic acids, and guidance in the choice of labels useful for various purposes, are known to one of ordinary skill in the art.

Differentiation: The process whereby relatively unspecialized cells (e.g., embryonic cells or stem cells) acquire specialized structural and/or functional features characteristic of mature cells. Similarly, “differentiate” refers to this process. Typically, during differentiation, cellular structure alters and tissue-specific proteins and properties appear. The term “differentiated neuronal cell” refers to cells expressing a protein characteristic of the specific neuronal cell type, exhibiting synaptic vesicle release, or having an electrophysiological characteristic of a neuronal cells (e.g., sustained bursts of action potentials).

Differentiation medium: A synthetic set of culture conditions with the nutrients necessary to support the growth or survival of microorganisms or culture cells, and which allows the differentiation of cells, such as neuronal stem cells.

Embryonic Stem (ES) Cells: Pluripotent cells isolated from the inner cell mass of the developing blastocyst, or the progeny of these cells. ES cells can be derived from any organism. ES cells can be derived from mammals, including mice, rats, rabbits, guinea pigs, goats, pigs, cows, monkeys and humans. Without being bound by theory, ES cells can generate a variety of the cells present in the body (e.g., bone, muscle, brain cells, etc.), provided they are exposed to conditions conducive to developing these cell types. Methods for producing murine ES cells can be found in U.S. Pat. No. 5,670,372, which is herein incorporated by reference. Methods for producing human ES cells can be found in U.S. Pat. No. 6,090,622, WO 00/70021 and WO 00/27995, which are herein incorporated by reference.

Exosome: Also known as a liquid phase extracellular vesicle (EV), an exosome is a membranous vesicle that is secreted by a cell and typically ranges in diameter from 10 to 150 nm (for example, exosome diameter can range from 30-100 nm). Generally, late endosomes or multivesicular bodies contain intralumenal vesicles which are formed by the inward budding and scission of vesicles from the limited endosomal membrane into these enclosed vesicles. These intralumenal vesicles are then released from the multivesicular body lumen into the extracellular environment, typically into a body fluid such as blood, cerebrospinal fluid or saliva, during exocytosis upon fusion with the plasma membrane. An exosome is created intracellularly when a segment of membrane invaginates and is endocytosed. The internalized segments which are broken into smaller vesicles and ultimately expelled from the cell contain proteins and RNA molecules such as mRNA and miRNA. Plasma-derived exosomes largely lack ribosomal RNA. Extracellular matrix-derived exosomes may include specific miRNA and protein components, and have been shown to be present in virtually every body fluid such as blood, urine, saliva, semen, and cerebrospinal fluid. Exosomes can express CD11c, CD63, CD81, and/or CD9, and, thus, can be CD11c+ and/or CD63+ and/or C81+ and/or CD9+.

Extracellular vesicles (EVs): Lipid bilayer-delimited particles that are released from a cell, but cannot replicate. EVs range in diameter from at least about 20-30 nm to at least about 10 μm but are typically smaller than 200 nm. EVs can carry proteins, nucleic acids, lipids, metabolites, and/or even organelles from the parent cell. Various types of EVs are included. For example, EVs vary in size, biogenesis pathway, cargo, cellular source, and function. Examples of EVs include exosomes, ectosomes, microvesicles (also known as microparticles), and apoptic bodies.

Expand: A process by which the number or amount of cells in a culture is increased due to cell division. Similarly, the terms “expansion” or “expanded” refers to this process. The terms “proliferate,” “proliferation,” or “proliferated” may be used interchangeably with the words “expand,” “expansion,” or “expanded.” Typically, during an expansion phase, the cells do not differentiate to form mature cells.

Expansion medium: A synthetic set of culture conditions suitable for the expansion of cells, such as neuronal stem cells. Tissue culture media generally include a carbon source, a nitrogen source and a buffer to maintain pH. In one embodiment, a medium contains a minimal essential media, such as DMEM, supplemented with various nutrients to enhance neuronal stem cell growth. Additionally, the minimal essential media may be supplemented with additives, such as horse, calf, or fetal bovine serum.

Hybridization: The ability of complementary single-stranded DNA, RNA, or DNA/RNA hybrids to form a duplex molecule (also referred to as a hybridization complex). Nucleic acid hybridization techniques can be used to form hybridization complexes between a nucleic acid probe (such as a detection probe), and the nucleic acid it is designed to target.

“Specifically hybridizable” and “specifically complementary” are terms that indicate a sufficient degree of complementarity such that stable and specific binding occurs between an oligonucleotide (or its analog) and the nucleic acid target (such as a DNA or RNA target, such as mRNA or miRNA). The oligonucleotide (such as a detection probe or bifunctional oligonucleotide) need not be 100% complementary (for example, it can be at least 90% complementary, such as at least 91%, at least 92%, at least 93%, at least 94%, at least 95%, at least 96%, at least 97%, at least 98%, or at least 99% complementary) to its target sequence (such as the target nucleic acid or anchor) to be specifically hybridizable; however, in some examples, the oligonucleotide is 100% complementary to a target sequence. In some examples, specific hybridization is also referred to herein as “specific binding.”

Hybridization conditions resulting in particular degrees of stringency will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11).

Lineage-specific: Characteristics of a cell that indicate the cell will become one of a limited number of related cell types or a particular cell type, such as a differentiated cell or a cell undergoing the process of differentiation into a specific cell type or a mature cell type.

MicroRNA: A small non-coding RNA that is about 17 to about 25 nucleotide bases in length, that post-transcriptionally regulates gene expression, typically by repressing target mRNA translation. In some examples, miRNA are characteristic of certain molecules or conditions, for example, miRNA that are characteristic of exosomes associated with certain conditions (such as exosomes associated with stem cells during various stages of cell differentiation). There are three forms of miRNAs, primary miRNAs (pri-miRNAs), premature miRNAs (pre-miRNAs), and mature miRNAs. Primary miRNAs (pri-miRNAs) are expressed as stem-loop structured transcripts of about a few hundred bases to over 1 kb. The pri-miRNA transcripts are cleaved in the nucleus by an RNase II endonuclease called Drosha that cleaves both strands of the stem near the base of the stem loop. Drosha cleaves the RNA duplex with staggered cuts, leaving a 5′ phosphate and 2 nucleotide overhang at the 3′ end. The cleavage product, the premature miRNA (pre-miRNA) is about 60 to about 110 nucleotides long with a hairpin structure formed in a fold-back manner. Pre-miRNA is transported from the nucleus to the cytoplasm by Ran-GTP and Exportin-5. Pre-miRNAs are processed further in the cytoplasm by another RNase II endonuclease called Dicer. Dicer recognizes the 5′ phosphate and 3′ overhang, and cleaves the loop off at the stem-loop junction to form miRNA duplexes. The miRNA duplex binds to the RNA-induced silencing complex (RISC), where the antisense strand is preferentially degraded and the sense strand mature miRNA directs RISC to its target site. It is the mature miRNA that is the biologically active form of the miRNA and is about 17 to about 25 nucleotides in length.

Nanorod: A nanoscale object, typically rod-shaped or cylindrical in shape, with each dimension typically being from about 1-300 nm, but not limited to this size range. In particular examples, nanorods are synthesized from magnetic materials, plasmonic materials, or a combination thereof.

Neuronal stem cell (NSC): An undifferentiated, multipotent, self-renewing neural cell. A NSC is a multipotent stem cell which is able to divide and, under appropriate conditions, has self-renewal capability and can terminally differentiate into neurons, astrocytes, and oligodendrocytes. Hence, the neural stem cell is “multipotent” because stem cell progeny have multiple differentiation pathways. A NSC is capable of self-maintenance, meaning that with each cell division, at least one daughter cell will also be, on average, a stem cell. Neural stem cells can be derived from tissues including, but not limited to brain and spinal cord. A “long term” NSC divides in culture for at least 15 cell divisions, such as at least 15, 20, 25, 30, 35, 40, 45 or 50 cell divisions. A long term NSC retains the properties of a neuronal stem cell and has the capacity to differentiate into neurons and glia in appropriate culture conditions in vitro.

NSCs can be obtained from a cadaver or living subject, including from fetal tissue and adult brain biopsies. NSCs can be produced from other stem cells, such as induced pluripotent stem cells or embryonic stem cells. NSCs can be autologous or heterologous to a recipient.

Neurological disorder: A disorder in the nervous system, including the central nervous system (CNS) and/or peripheral nervous system (PNS). Examples of neurological disorders include Parkinson's disease, Huntington's disease, Alzheimer's disease, severe seizure disorders including epilepsy, familial dysautonomia, as well as injury or trauma to the nervous system, such as neurotoxic injury, or disorders of mood and behavior such as addiction, schizophrenia and amyotrophic lateral sclerosis.

Neuronal disorders also include Lewy body dementia, multiple sclerosis, epilepsy, cerebellar ataxia, progressive supranuclear palsy, amyotrophic lateral sclerosis, affective disorders, anxiety disorders, obsessive compulsive disorders, personality disorders, attention deficit disorder, attention deficit hyperactivity disorder, Tourette Syndrome, Tay Sachs, Nieman Pick, and other lipid storage and genetic brain diseases and/or schizophrenia

Neurodegenerative disorder: An abnormality in the nervous system of a subject, such as a mammal, in which neuronal integrity is compromised. Without being bound by theory, neuronal integrity can be compromised when neuronal cells display decreased survival or when the neurons can no longer propagate a signal. Specific, non-limiting examples of a neurodegenerative disorder are Alzheimer's disease, Pantothenate kinase associated neurodegeneration, Parkinson's disease, Huntington's disease (Dexter et al., Brain 114:1953-1975, 1991), HIV encephalopathy (Miszkziel et al., Magnetic Res. Imag. 15:1113-1119, 1997), and amyotrophic lateral sclerosis.

A “neurodegenerative-related disorder” is a disorder such as speech disorders that are associated with a neurodegenerative disorder. Specific non-limiting examples of a neurodegenerative-related disorders include, but are not limited to, palilalia, tachylalia, echolalia, gait disturbance, perseverative movements, bradykinesia, spasticity, rigidity, retinopathy, optic atrophy, dysarthria, and dementia.

Nucleic acid: A molecule formed by nucleotides, such as a polynucleotide, for example a polynucleotide sequence (such as a linear sequence) of any length. Therefore, a nucleic acids include oligonucleotides and gene sequences found in chromosomes. An “oligonucleotide” is a plurality of joined nucleotides joined by native phosphodiester bonds. An oligonucleotide is typically a polynucleotide of between 6 and 300 nucleotides in length. An oligonucleotide analog refers to moieties that function similarly to oligonucleotides but have non-naturally occurring portions. For example, oligonucleotide analogs can contain non-naturally occurring portions, such as one or more altered sugar moieties or inter-sugar linkages, such as a phosphorothioate oligodeoxynucleotide. Functional analogs of naturally occurring nucleic acids can bind to RNA or DNA, and include peptide nucleic acid (PNA) or locked nucleic acid (LNA) molecules.

Pharmaceutically acceptable carriers: The pharmaceutically acceptable carriers useful in the claimed pharmaceutical preparations are conventional. Remington's Pharmaceutical Sciences, by E. W. Martin, Mack Publishing Co., Easton, Pa., 22nd Edition, 2013, describes compositions and formulations suitable for pharmaceutical delivery of the agents herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically-neutral carriers, pharmaceutical preparations to be administered can contain minor amounts of non-toxic auxiliary substances, such as wetting or emulsifying agents, preservatives, and pH buffering agents and the like, for example sodium acetate or sorbitan monolaurate.

Preventing or treating a disease: “Preventing” a disease refers to inhibiting the development of a disease, for example in a person who is known to have a predisposition to a disease. An example of a person with a known predisposition is someone with a history of a disease in the family, or who has been exposed to factors that predispose the subject to a condition. “Treatment” refers to a therapeutic intervention that ameliorates or reduces a sign or symptom of a disease or pathological condition after it has begun to develop.

Peptide: Any chain of amino acids, regardless of length (thus encompassing oligopeptides, peptides, and proteins) or post-translational modification (for example, glycosylation, phosphorylation, or acylation). A polypeptide encompasses also a protein precursor as well as mature proteins and fragments thereof.

Sample (or biological sample): A biological specimen. For example, a sample can contain biological bodies (such as cells, EVs, or exosomes) or molecules (such as nucleic acids or peptides), for example, obtained from a subject. Examples include, but are not limited to cells, cell culture medium, cell lysates, exosomes, EVs, peripheral blood, plasma, serum, cerebrospinal fluid, urine, saliva, tissue biopsy (such as a tumor biopsy or lymph node biopsy), fine needle aspirate, surgical specimen, bone marrow, amniocentesis samples, cell culture media (such as from stem cell culture), and autopsy material.

Specific binding: Binding of an agent substantially or preferentially only to a defined target such as a defined protein, peptide, oligonucleotide, DNA, RNA, mRNA, miRNA, or portion thereof. Thus, a nucleic acid-specific binding agent binds substantially only to a defined nucleic acid, (such as a target sequence in a target nucleic acid) and does not substantially bind to any other nucleic acid. In some examples, specific binding includes the hybridization of one nucleic acid molecule to a target (e.g., a labeled aptamer or molecular beacon, such as a labeled probe to a target nucleic acid or peptide). For example, a nucleic acid molecule specifically binds another nucleic acid molecule if a sufficient amount of the nucleic acid molecule forms base pairs or is hybridized to its target nucleic acid molecule to permit detection of that binding. In some examples, a nucleic acid molecule specifically binds to another nucleic acid molecule if the two nucleic acids are at least 90% complementary to one another (such as at least 91%, 92%, 93%, 94%, 95%, 96%, 97%, 98%, 99%, or 100% complementary). In specific, non-limiting examples, specific binding is between nucleic acids (such as between an miRNA and a single-stranded nucleic acid, such as a hairpin DNA).

In some examples, specific binding is between an antibody (immunoglobulin molecules and immunologically active portions of immunoglobulin molecules, for instance, molecules that contain an antigen binding site) and an antigen. Naturally occurring antibodies (for example, IgG, IgM, and IgD) include four polypeptide chains, two heavy (H) chains and two light (L) chains inter-connected by disulfide bonds; fragments of a naturally occurring antibody are also intended to be designated by the term “antibody” (such as (i) a Fab fragment consisting of the VL, VH, CL, and CH1 domains; (ii) an Fd fragment consisting of the VH and CH1 domains; (iii) an Fv fragment consisting of the VL and VH domains of a single arm of an antibody, (iv) a dAb fragment (Ward et al., Nature 341:544-546, 1989) which consists of a VH domain; (v) an isolated complementarity determining region (CDR); and (vi) a F(ab′)2 fragment, a bivalent fragment comprising two Fab fragments linked by a disulfide bridge at the hinge region). In specific, non-limiting examples, the antibody binds surface accessible proteins on an extracellular vesicle (such as exosomes).

Subject: Human and non-human animals, including all vertebrates, such as mammals and non-mammals, such as non-human primates, mice, rabbits, sheep, dogs, cats, horses, cows, chickens, amphibians, and reptiles. In some embodiments of the described methods, the subject is a human.

Target nucleic acid molecule: A defined region or particular portion of a nucleic acid molecule, for example a DNA or RNA (e.g., mRNA or miRNA) of interest. In an example, where the target nucleic acid molecule is a target miRNA, such a target can be defined by its specific sequence or function, by its name, or by any other means that uniquely identifies it from among other nucleic acids. Exemplary target nucleic acid molecules, such as miRNA, are disclosed herein.

Therapeutically effective amount: A quantity of a specific substance, such as stem cells, sufficient to achieve a desired effect in a subject being treated. When administered to a subject, a dosage will generally be used that will achieve a desired effect.

Treating, Treatment, and Therapy: Any success or indicia of success in the attenuation or amelioration of an injury, pathology or condition, including any objective or subjective parameter, such as abatement, remission, diminishing of symptoms, or making the condition more tolerable to the patient, slowing in the rate of degeneration or decline, making the final point of degeneration less debilitating, or improving a subject's physical or mental well-being. The treatment may be assessed by objective or subjective parameters; including the results of a physical examination, neurological examination, or psychiatric evaluations.

### II. Devices and Kits for Isolating and Characterizing Vesicles

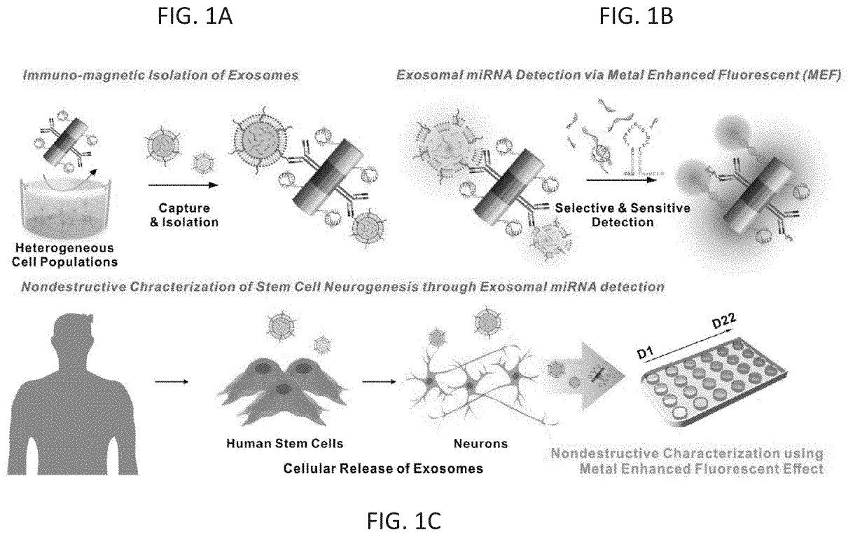

Disclosed herein are devices (for example, multifunctional magneto-plasmonic nanorods) and methods of isolating, detecting, and/or characterizing exosomes from a sample. In some embodiments, the devices and methods are utilized for characterizing stem cell differentiation in a non-destructive manner (see, e.g., FIGS. 1A-1C).

Disclosed herein are devices (e.g., nanodevices, such as nanorods) for isolating and/or characterizing biological bodies (such as cells or vesicles, for example, exosomes) or molecules (for example, nucleic acids or peptides). The disclosed nanorods are characterized by their size (the scale of nanometers to low micrometers) and shape (e.g., a cylindrical shape). The nanorods further include plasmonic and magnetic properties. In some examples, the magneto-plasmonic composition of the nanorods facilitates the isolation and characterization of biological bodies and/or molecules.

In some embodiments, the nanorods include a cylindrical body with end portions and a center portion (e.g., a portion of the nanorod that extends throughout the diameter, but the length of which does not reach either end of the cylinder). The center can include a smaller, greater, or equivalent length compared to one or more of the ends. The end portions (e.g., portions that flank the center portion) can be the same or different lengths. In some examples, the ends are both the same length and each have the same or a smaller length than the center.

Generally, the diameter of the nanorods is in the nanometer scale. In some examples, the diameter of the nanorod is at least about 1 nm, 2 nm, 5 nm, 10 nm, 20 nm, 30 nm, 40 nm, 50, nm, 60 nm, 70 nm, 80 nm, 90 nm, 100 nm, 115 nm, 130 nm, 150 nm, 175 nm, 200 nm, 250 nm, 300 nm, 400 nm, 500 nm, or 750 nm, or about 1-800 nm, 1-500 nm, 1-250 nm, 1-150 nm, 30-150 nm, 30-120 nm, 50-100 nm, 100-400 nm, or 200-300 nm. In one non-limiting example, the diameter of the nanorod is about 250, 265, or 280 nm.

The length of the nanorods is also generally in the nanometer range but can reach the lower micrometer range. In some examples, the length of the nanorods is at least about 50 nm, 100 nm, 200 nm, 300 nm, 400 nm, 500 nm, 600 nm, 700 nm, 800 nm, 900 nm, 1 μm, 1.1 μm, or 1.25 μm, or about 50 nm-1.25 μm, 250 nm-1.1 μm, 500 nm-1.1 μm, 750 nm-1.1 μm, or 900 nm-1.1 μm, or about 1 μm. In one non-limiting example, the length of the nanorod is about 750 nm. Further, the length of the nanorods that can be considered the center portion or the end portions can vary. In some examples the length of the center portion of the nanorod is at least about 50 nm, 100 nm, 200 nm, 300 nm, 400 nm, 500 nm, 600 nm, 700 nm, 800 nm, 900 nm, or 1μm, or about 250 nm-1.1 p.m, 500 nm-1.1 p.m, 500 nm-900 nm, or 600 nm-800 nm, or about 250 nm or 750 nm. In other examples, the length of one or both of the end or flanking portions is at least about 10 nm, 20 nm, 50 nm, 100 nm, 200 nm, 300 nm, 400 nm, or 500 nm, or about 10 nm-500 nm, 50 nm-250 nm, 50 nm-200 nm, or 100 nm-200 nm, or about 150 nm. In some examples, both end portions are each about 150 nm or 250 nm long. In additional embodiments, the nanorods include various aspect ratios (for example, the ratio of length to the width, such as the width of the diameter), such as at least about 1.5:1, 2:1, 2.5:1, 3:1, 3.5:1, 4:1, 4.5:1, 5:1, 7.5:1, or 10:1, or about 1.5:1-10:1, 2-5:1, or 2.5-3.5:1, or about 3:1.

The nanorods disclosed herein include at least one portion (such as one or both end portions) including or consisting of a plasmonic material. In some examples, a plasmonic composition forms the body of the nanorod throughout the diameter or at an outer layer of the nanorods at one or more of the center or ends. Examples of plasmonic compositions include gold, silver, and platinum. Any of these plasmonic compositions may be used alone or as a mixture with other materials, such as a mixture with at least 1%, 5%, 10%, 15%, 20%, 25%, 30%, 40%, 50%, 60%, 70%, 75%, 80%, 90%, 95%, or 99% or about 1%-10%, 1-25%, 20%-80%, 20%-50%, 50%-99%, 50%-75%, 75%-99%, 80%-99%, 90%-99%, or 95%-99% of one or more plasmonic compositions in a mixture with other materials. In some examples, the plasmonic composition is used in a mixture with at least 1, at least 2, at least 3, at least 4, at least 5, at least 8, at least 10, at least 12, at least 15, at least 18, or at least 25 or about 1-2, 1-5, 1-10, 1-15, or 1-25 other materials. In some examples, gold is used as a plasmonic composition. In some examples, the plasmonic composition forms the body of the nanorods at one or more ends (for example, the plasmonic composition can form the entire diameter of one or more of the nanorods ends). In specific, non-limiting examples, the nanorods are formed by gold at both ends. In specific, non-limiting examples, the nanorods are formed by gold at both ends, which are each about 150 nm long.

The nanorods disclosed herein also include at least one portion (such as a center portion) including or consisting of a magnetic composition. In some examples, a magnetic composition forms the body of the nanorod at one or more portions (for example, in the center of the nanorod or at one or both ends) and/or at an outer layer.

Examples of magnetic compositions include nickel, iron, and cobalt. Any of these magnetic compositions can be used alone or as a mixture with other materials. Any of these magnetic compositions may be used alone or as a mixture with other materials, such as a mixture with at least 1%, 5%, 10%, 15%, 20%, 25%, 30%, 40%, 50%, 60%, 70%, 75%, 80%, 90%, 95%, or 99% or about 1%-10%, 1-25%, 20%-80%, 20%-50%, 50%-99%, 50%-75%, 75%-99%, 80%-99%, 90%-99%, or 95%-99% of one or more magnetic compositions in a mixture with other materials. In some examples, the magnetic compositions is used in a mixture with at least 1, at least 2, at least 3, at least 4, at least 5, at least 8, at least 10, at least 12, at least 15, at least 18, or at least 25 or about 1-2, 1-5, 1-10, 1-15, or 1-25 other materials. In some examples, nickel is used in the magnetic compositions. In other examples, iron oxide or iron oxide nanoparticles are used in the magnetic compositions. In some examples, the magnetic composition forms the body of the nanorods at least in the center (for example, the magnetic composition can form the entire diameter of the nanorod center). In other examples, the nanorods are formed by nickel in the center.

In specific, non-limiting examples, the disclosed nanorods include end portions formed by gold flanking a center portion formed by nickel, such as a nanorod with two ends formed by gold, each about 150 nm, 248 nm, or 250 nm long, and a center formed by nickel, about 250 nm or 750 nm long.

The nanorods disclosed herein also include an immunoactive material, such as one or more immunoactive macromolecules (e.g., an antibody or antigen binding portion thereof or an aptamer) associated with at least a portion of the nanorod. The immunoactive material can aid in identifying and/or isolating biological bodies (such as vesicles or cells) or molecules of interest (such as nucleic acids or peptides). The immunoactive material in some examples is coupled to the surface of the nanorods, such as along the entire surface, at the center, or to the surface along the center of the nanorods, in which the center of the nanorod at one or both ends of the nanorods. In some examples, the immunoactive material is coupled to the surface along the center portion of the nanorods (e.g., is coupled to the magnetic composition). In specific, non-limiting examples, the immunoactive material is coupled to a magnetic composition, such as nickel.

In example embodiments, the immunoactive material is at least one antibody or portion thereof specific for biological bodies or proteins of interest. In examples, at least one antibody or portion thereof can specifically bind one or more antigens. In specific, non-limiting examples, at least one antibody or portion thereof can bind proteins or peptides expressed or accessible at the surface of cells or biological bodies, such as exosomes. For example, antibodies that specifically bind surface-expressed or surface-accessible proteins or peptides can include naturally occurring antibodies or fragments of a naturally occurring antibody (such as (i) an Fab fragment consisting of the VL, VH, CL, and CH1 domains; (ii) an Fd fragment consisting of the VH and CH1 domains; (iii) an Fv fragment consisting of the VL and VH domains of a single arm of an antibody, (iv) a dAb fragment (Ward et al., Nature 341:544-546, 1989) which consists of a VH domain; (v) an isolated complementarity determining region (CDR); and (vi) an F(ab′)2 fragment, a bivalent fragment comprising two Fab fragments linked by a disulfide bridge at the hinge region. In some examples, the antibody or antigen binding fragment specifically binds to proteins or peptides expressed or accessible at the surface of exosomes, such as CD11c, CD63, CD81, CD82, and/or CD9. Therefore, in order to identify and isolate exosomes, the at least one antibody or antigen binding fragment specifically binds to CD11c, CD63, CD81, CD9, or any combination thereof. In specific, non-limiting examples, the antibody specifically binds to CD63. However, additional proteins or peptides expressed on the surface of exosomes can be identified, for example, depending on the type of cell or tissue source of the exosomes. In specific, non-limiting examples, the at least one antibody is coupled to the surface along the center of the nanorods. In specific, non-limiting examples, the at least one antibody is coupled to the surface along the center of the nanorods, in which the center of the nanorods is formed by a magnetic composition, such as nickel.

In other examples, the immunoactive material is an aptamer, such as a nucleic acid that can selectively bind to a specific target such as a protein, peptide, carbohydrate, or other small molecule. In one non-limiting example, the aptamer binds to surface protein CD63 (see, e.g., Jin et al., Anal. Chem., 90, 24, 14402-14411, 2018, incorporated herein by reference).

The disclosed nanorods also include at least one detectable molecule capable of selectively binding a target nucleic acid and including at least one detectable label (such as a molecular beacon). The at least one detectable molecule is coupled to the surface of the nanorods, such as along the entire surface, at the center, or at one or both ends of the nanorods. In some examples, the at least one detectable molecule is coupled to the surface along the end portions of the nanorods. In specific, non-limiting examples, the at least one detectable molecule is coupled to the surface along the end portions of the nanorods, in which the end portions of the nanorods are formed by a plasmonic composition, such as gold.

In example embodiments, the at least one detectable molecule includes a portion including at least one nucleic acid molecule specific for a target nucleic acid of interest. The at least one detectable molecule can form a variety of lengths. For example, the at least one detectable molecule can be at least about 4 nm, 5 nm, 6 nm, 7 nm, 8 nm, 9 nm, 10 nm, 11 nm, 12 nm, 13 nm, 14 nm, 15 nm, 16 nm, 17 nm, or 20 nm, or about 4 nm-20 nm, 6 nm-16 nm, 6 nm-12 nm, 6 nm-10 nm, 7 nm-9 nm, or 8 nm-9nm, or about 8.5 nm long. In some examples, the at least one detectable molecule includes a nucleic acid at least about 15 bp, 18 bp, 20 bp, 21 bp, 22 bp, 23 bp, 24 bp, 25 bp, 26 bp, 27 bp, 28 bp, 29 bp, 30 bp, 32 bp, 35 bp, 38 bp, 40 bp, 42 bp, 45 bp, 49 bp, 55 bp, or 60 bp, or about 15 bp-60 bp, 15 bp-45 bp, 15 bp-30 bp, 20 bp-30 bp, 22 bp-28 bp, or about 25 bp long (such as a DNA hairpin about 25 bp long). In examples, the at least one nucleic acid molecule can specifically bind one or more target miRNAs. For example, the detectable molecule can be a hairpin DNA complementary to a target miRNA of interest. In specific, non-limiting examples, the at least one nucleic acid specifically binds target miRNA(s) expressed by biological bodies in a biological condition, such as target miRNA(s) expressed by exosomes during stem cell differentiation or in a disease state. Examples of target miRNAs expressed by exosomes include miRNA-124 and miRNA-449a.

In example embodiments, the at least one detectable molecule also includes a detectable label. A variety of labels are possible. In specific, non-limiting examples, the label is a fluorophore. Various fluorophores can be used. Examples of fluorophores include xanthene derivatives, such as fluorescein, rhodamine, Oregon green, eosin, and Texas red; cyanine derivatives, such as cyanine, indocarbocyanine, oxacarbocyanine, thiacarbocyanine, and merocyanine; squaraine derivatives and ring-substituted squaraines, including seta and square dyes, squaraine; rotaxane derivatives, such as setau dyes; naphthalene derivatives (dansyl and prodan derivatives); coumarin derivatives; oxadiazole derivatives, such as pyridyloxazole, nitrobenzoxadiazole and benzoxadiazole; anthracene derivatives, such as anthraquinones, including draq5, draq7 and cytrak orange; pyrene derivatives, such as cascade blue, etc.; oxazine derivatives, such as Nile red, Nile blue, cresyl violet, oxazine 170, etc.; acridine derivatives, such as proflavin, acridine orange, acridine yellow, etc.; arylmethine derivatives, such as auramine, crystal violet, and malachite green; and tetrapyrrole derivatives, such as porphin, phthalocyanine, and bilirubin. In specific, non-limiting examples, the fluorophore can be a fluorescein.

In particular examples, the detectable molecule is a molecular beacon. In some examples, the molecular beacon includes a portion that is complementary to a target nucleic acid (such as a target miRNA) and portions at each end that are complementary to one another. The complementary portions hybridize to one another, forming a hairpin structure. In addition, the molecular beacon includes a fluorophore at one end (such as the 5′ end) and a quencher at the other end (such as the 3′ end). When the ends are hybridized to one another, the fluorophore is quenched by the close proximity of the quencher. If the portion complementary to the target nucleic acid hybridizes to the target, the loop is opened and fluorescent emission of the fluorophore is detectable, because the quencher is no longer in close proximity to the fluorophore. In other examples, an aptamer can be used in place of a molecular beacon. Alternatively, an aptamer can be used instead of or as a molecular beacon. An exemplary aptamer binds to the target and changes its conformation to generate a fluorescent signal (see, e.g., Jo et al., Anal. Chem., 90(1): 716-722, 2018, incorporated herein by reference).

Further contemplated herein are kits that include a nanorod disclosed herein. For example, the kit may include magneto-plasmonic nanorods for isolating and/or identifying biological bodies (such as cells or vesicles) and/or molecules (such as nucleic acids or peptides) of interest. In example embodiments, the kit includes nanorods coupled to immunoactive molecules (such as antibodies), nanorods coupled to detectable molecules (such as molecular beacons), or both. Kits can further include one or more containers including some or all of the reagents (such as buffers, immunomagnetic isolation reagents) for carrying out isolation, detection, and or characterization of exosomes. Instructions for use can also be included.

### III. Methods Of Making Disclosed Nanorods

Methods of producing the disclosed nanorods are provided. Typically, the disclosed composition is made by synthesizing an initial portion or section of the nanorods, such as the one of the ends and then sequentially adding the next sections (such as the center and the other end) onto that first section. For example, nanorod compositions may be made by first synthesizing a first plasmonic end, and then synthesizing each subsequent portion onto the first end (such as synthesizing a magnetic center section followed by a plasmonic second end section onto the plasmonic first end). The sections typically are made using an anodized template methods (see, for example, Martin, C. R., Science, 266:1961-1966, 1994; Lee, S. A. et al., J. Phys. Chem.

C, 116: 18388-18393, 2012; Banholzer, M. J. et al., Nat. Protoc., 4:838-848, 2009, all of which are incorporated herein by reference in their entireties) with the final product being a nanorod with a magnetic center and plasmonic ends (a magneto-plasmonic nanorod). In some examples, the nanorod is synthesized on an anodized aluminum oxide template, as described in Example 1.

In example embodiments, the magneto-plasmonic nanorods are coupled with immunoactive materials (such as antibodies), detectable molecules (such as molecular beacons), or both. The immunoactive materials (such as antibodies) or detectable molecules can be coupled to the magneto-plasmonic nanorods in a variety of ways.

In specific, non-limiting examples, the detectable molecules are coupled only with the plasmonic portion of magneto-plasmonic nanorods (such as one or both ends of the nanorod). In some examples, the plasmonic portion (such as a gold portion) of the magneto-plasmonic nanorods is coupled to one or more detectable molecules through covalently binding detectable molecules with a surface thiol group to the plasmonic portion of the nanorod. A variety of detectable molecules can include a thiol group. For example, nucleic acids and peptides can be synthesized with or functionalized to include a thiol group, and some peptides include an endogenous thiol. Reducing agents may be used during coupling to facilitate reaction of the plasmonic material (such as gold) with the thiol-containing detectable molecule. In specific, non-limiting examples, the detectable molecule is a thiol-containing nucleic acid. In additional specific, non-limiting examples, the detectable molecule is a thiol-containing single-stranded nucleic acid. In further specific, non-limiting examples, the detectable molecule is a thiol-containing hairpin DNA (such as a molecular beacon). One of ordinary skill in the art can identify and utilize other methods to couple the plasmonic material to a detectable molecule.

In some examples, the immunoactive materials are antibodies or an antigen binding fragment thereof. In some embodiments, the detectable molecules are coupled only with the magnetic portion of magneto-plasmonic nanorods (such as the center portion of the nanorod). In specific, non-limiting examples, antibodies or antigen binding fragments are coupled with magneto-plasmonic nanorods after the plasmonic portion of the nanorods is coupled with a detectable molecule. For example, a solution of antibodies or antigen binding fragments can be incubated with detectable molecule-containing nanorods, thus, facilitating binding of the antibodies or antigen binding fragments at the non-plasmonic, or magnetic, portion of the nanorods. In some non-limiting embodiments, a carboxyl group of the antibody or antigen binding fragment is coupled to the magnetic portion of the nanorods. In examples, an inactive protein can be added thereafter, for example, to reduce non-specific binding to the nanorods after the solution of antibodies or antigen binding fragments is removed.

### IV. Methods of Isolating or Purifying Target Molecules or Components

Provided herein are methods of isolating or purifying target molecules (such as nucleic acids or proteins) or components (such as vesicles or cells) of interest from a sample. In examples, the components (such as vesicles or cells) or target molecules for isolation re from a sample from a subject or a cell culture sample.

The methods include contacting a sample with any of the nanorods disclosed herein under conditions sufficient for the nanorod(s) to bind to the target molecule or component. In some examples, the sample includes, but is not limited to cells, cell lysates, peripheral blood, serum, plasma, urine, saliva, tissue biopsy (such as a tumor biopsy or lymph node biopsy), fine needle aspirate, surgical specimen, bone marrow, amniocentesis samples, cell or tissue culture media (such as from a stem cell culture), cerebrospinal fluid, and autopsy material. In specific, non-limiting examples, the methods are used to isolate exosomes from a biological sample, such as cell or tissue culture media (such as from stem cell culture) or a sample from a subject (such as a blood, cerebrospinal fluid, urine, or biopsy sample).

In example embodiments, the nanorods bind to a component (including cells, vesicles, organelles) or molecule of interest of interest in the sample. In some examples, the nanorods include immunoactive molecules that specifically bind the component (including cells, vesicles, organelles, or exosomes) or molecule of interest. In examples, the nanorods include antibodies that specifically bind the component or molecule of interest. In specific, non-limiting examples, the component of interest is a vesicle. In specific, non-limiting examples, the component of interest is an exosome. In specific, non-limiting examples, the magneto-plasmonic nanorods include one or more antibodies (or fragments thereof) that specifically bind one or more proteins expressed at the surface of exosomes, such as CD11c, CD63, CD81, CD82, and/or CD9.

Following binding of the nanorods to the target component or molecule of interest, the nanorods are isolated. In example embodiments, the magnetic properties of the nanorods are used for isolation and/or purification. For example, the methods include applying a magnetic field to the sample containing the nanorods bound to the component or molecule of interest, immobilizing the nanorod complex. One exemplary method of isolating exosomes using the disclosed nanorods is provided in FIG. 1A. In additional examples, the immobilized nanorods with the bound component or molecule of interest is further subject to a variety of purification steps, such as washing and resuspension in a solution (for example, a buffered-solution, such as phosphate-buffered saline). The component and/or molecule of interest may further be detected, identified, or analyzed.

### V. Methods of Detecting a Target Molecule

Further disclosed herein are methods of detecting a target molecule (such as a target nucleic acid or peptide). The methods include contacting a sample with any of the nanorods disclosed herein under conditions sufficient for the nanorod(s) to bind to the target molecule via the detectable molecule coupled to the plasmonic portion of the nanorod. In some examples, the target molecule is enclosed within a component (such as a cell, vesicle or exosome) bound to the nanorod (which may be isolated or purified as described above). Therefore, in some examples, the methods include lysing the component to provide the nanorod access to the target molecule to which is can specifically bind. A variety of lysis techniques can be used. In some examples, a micelle-forming solution, such as a detergent-containing solution, is utilized. For example, non-ionic detergent (such as TWEEN® 20, TWEEN® 80, BRIJ™-35, BRIJ™-58, TRITON™ X-100, TRITON™ X-114, NP-40, octyl glucoside, and/or octyl thioglucoside); anionic detergent (such as sodium dodecyl sulfate, SDS), and/or zwitterionic detergents (such as CHAPS and/or CHAPSO) is used. In specific examples, a non-ionic detergent, such as TWEEN® 20 is used.

The methods also include detecting the target molecule. In example embodiments, the detectable molecule coupled to the plasmonic portion of the nanorod includes a label, such as a fluorophore. Thus, detecting the target molecule specifically bound to the nanorod includes applying an excitation wavelength and measuring or detecting an emission wavelength. Excitation and emission properties of fluorophores are known by one of ordinary skill in the art. In specific, non-limiting examples, the excitation wavelength is at least about 485-500 nm or about 490 or 494 nm (for example, where the fluorophore is fluorescein). In specific, non-limiting examples, the emission wavelength is at least about 510-525 nm or about 512 or 520 nm (for example, where the fluorophore is fluorescein). Further, the intensity of the emission wavelength can be modified based on a variety of factors, such as level of intensity of the excitation wavelength and environment of the fluorophore. For example, the fluorophore can be more or less accessible to light energy, such as where a fluorophore is sequestered in a molecule (such as a peptide or nucleic acid).

In some examples, the methods can be used to detect modifications in the environment of a detectable molecule, such as a fluorescently labeled detectable molecule. For example, a fluorescently labeled detectable molecule can change conformation upon binding of a target molecule and, thus, change accessibility to a fluorophore when it binds a macromolecule (such as the opening of a hairpin DNA upon binding a complementary sequence). In other examples, binding of a target molecule changes the spatial relationship of the fluorophore and a quencher molecule in the detectable molecule, increasing or decreasing the fluorophore emission intensity. Thus, upon binding a macromolecule, the fluorophore portion of the detectable molecule emits a measurably different wavelength intensity compared with an unbound detectable molecule. In further examples, the signal intensity the fluorophore can be increased by plasmonic properties of the nanorods, for example, where fluorescent molecular beacons are coupled at the ends of nanorods formed by a gold composition. For example the increase in fluorescence intensity due to plasmonic properties can be at least about 1-fold, 2-fold, 5-fold, 7-fold, 8-fold, 9-fold, 10-fold, 11-fold, 12-fold, 13-fold, 14-fold, 15-fold, 18-fold, 20-fold, 30-fold, 40-fold, 50-fold, 10²-fold, 10³-fold, 10⁴-fold, 10⁵-fold, 10⁶-fold, or about 1-10⁶-fold, 5-15-fold, 5-20-fold, 8-15-fold, 10-15-fold, 12-13-fold, 10-10⁶, 10-10⁵, 10-10⁴, 10-10³, or 10-10² or about 12-fold or 13-fold.

In specific, non-limiting examples, the fluorescently labeled detectable molecule is a single-stranded DNA that forms a hairpin, which opens and binds a target complementary nucleic acid (such as miRNA). In specific, non-limiting examples, the fluorescently labeled hairpin increases in emission intensity upon binding a complementary DNA (such as an miRNA). In specific, non-limiting examples, a fluorescently labeled detectable molecule is coupled to a plasmonic portion of the nanorods, which can enhance a fluorescent signal. In specific, non-limiting examples, the target molecule of interest is a nucleic acid. In specific, non-limiting examples, the target molecule of interest is an miRNA (such as miR-124 or miR449a). One exemplary method of detecting a target miRNA in exosomes using the disclosed nanorods is provided in FIG. 1B.

### VI. Methods of Treatment

The methods disclosed herein can be used for a variety of applications. For example, the methods can be used to identify a specific disease or stage of a disease (such as a neurodegenerative disease or cancer), or the methods can be used to identify the level of cell differentiation (such as stem cell differentiation). Thus, in some embodiments, the methods can be used to treat a disease or disorder in a subject.

In some examples, the methods are used to identify and treat a specific disease or disease state. For example, various components and/or molecules are known by persons skilled in the art to be associated with certain diseases or disease states. In some examples, the methods are used to identify exosomes associated with one or more specific miRNAs that are associated with certain diseases or disease states. In specific, non-limiting examples, the methods include identifying and isolating exosomes using the nanorods herein and further measuring one or more specific miRNA expressed by exosomes, for example, with a fluorescently labeled hairpin DNA that includes a sequence complementary to the specific miRNA. Thus, in some examples, the methods include measuring differential expression of one or more miRNAs compared with a control (for example, a subject which does not have the disease of interest). The methods may further include administering a treatment to the subject for the identified disease.

In some examples, the specific disease or disease state is a neurodegenerative disease. A variety of neurodegenerative diseases can be included. In some examples, the neurodegenerative disease is Alzheimer's disease, Pantothenate kinase associated neurodegeneration, tauopathies, Parkinson's disease, Huntington's disease, HIV, transmissible spongiform encephalopathy, or amyotrophic lateral sclerosis. Thus, in examples, the magneto-plasmonic nanorods disclosed herein include an antibody (or portion thereof) for identifying and isolating exosomes as well as an labeled detectable molecule (such as a fluorescently labeled hairpin DNA that includes a sequence complementary to one or more miRNAs associated with one or more neurodegenerative diseases). In some examples, the miRNA includes one or more of let-7b, miR-128, miR-139-5p, miR-146a, miR-320, miR-328, miR-342-3p, miR-342-3p, miR-494, and miR-146a (for example, as associated with prion-mediated neurodegenerative diseases, such as transmissible spongiform encephalopathies); miR-9, miR-124, miR-125b, miR-128, miR-132, miR-219 , miR-9, miR-29a , miR-106a, miR-107, miR-298, miR-17-5p, miR-20, miR-106b, miR-9, miR-125b, miR-146a, miR-146a, miR-101, miR-9, miR-29a, miR-15, miR-107, miR-29a, miR-29b, miR-212, miR-424, miR-125b, and miR-146a (for example, as associated with Alzheimer's disease); miR-133b, miR-7, miR-184, miR-34b, and miR-34c (for example, as associated with Parkinson's disease); miR-128, miR-15, miR-9, miR-124, miR-132, miR-137, miR-132, and miR-212 (for example, as associated with tauopathies); miR-520f-3p, miR-135b-3p, miR-4317, miR-3928-5p, miR-8082, miR-140-5p, miR-509-3-5p, miR-6516-5p, miR-455-3p, miR-6838-3p, miR-552-5p, miR-761, miR-4659a-5p, miR-4781-5p, miR-4462, miR-132-5p, miR-6818-5p, miR-34c-3p, miR-4724-3p, miR-4307, miR-6874-5p, miR-5581-3p, miR-6807-5p, miR-922, and miR-1322 (for example, as associated with Huntington's disease); miR132-5p, miR132-3p, miR143-5p, miR143-3p, let7B-5p, miR39-3p, miR338-3p, miR1825, miR1915-3p, miR3665, miR4530, miR4745-5p, miR39-3p, miR3665 miR4530, miR4745-5p, miR106B, miR206, miR1234-3p, miR1825, miR39-3p, miR143-3p, miR206, miR374B-5p, miR17-5p, miR24, miR223-3p, miR142-3p, miR1249-3p, miR39-3p, miR27A-3p, miR4649-5p, miR4299, miR4516, miR424, miR206, miR16-5p, miR206/miR338-3p, miR9/miR129-3p, miR335-5p/miR338-3p, miR143-5p, miR574-5p, miR132-5p, miR132-3p, miR143-3p, miR338-3p , miR181A-5p, let7A-5p, let7B-5p, let7F-5p, miR15b-5p, miR21-5p, miR195-5p, miR148A-3p, miR39-3p, miR608, miR328-3p, miR39-3p, miR30A-5p, let7A-5p, let7D-5p, let7F-5p, let7G-5p, let7I-5p, miR15A-5p, miR15B-5p, miR151A-5p, miR151B, miR16-5p, miR22-3p, miR23A-3p, miR26A-5p, miR26B-5p, miR27B-3p, miR28-3p, miR30B-5p, miR30C-5p, miR93-5p, miR103A-3p, miR106B-3p, miR128-3p, miR130A-3p, miR130B-3p, miR144-5p, miR148A-3p, miR148B-3p, miR182-5p, miR183-5p, miR186-5p, miR221-3p, miR223-3p, miR342-3p, miR425-5p, miR451A, miR532-5p, miR550A-3p, and miR584-5p (such as associated with ALS).

In some embodiments, the methods include measuring differential expression of one or more neurodegenerative disease-associated miRNAs, for example, compared with a control subject, which does not have a neurodegenerative disease, thereby identifying a neurodegenerative disease in the subject. The methods may further include treating the subject for the neurodegenerative disease. For example, the methods can include administering symptomatic treatment, disease modifying treatment, or palliative care, such as one or more of anti-cholinesterase inhibitors, dimebolin, hypothalamic proline-rich peptide, antioxidants, and anti-inflammatory agents to the subject. In particular examples, the neurodegenerative disease is a neuroinflammation-related neurodegenerative disease, such as Alzheimer's disease, Parkinson's disease, or ALS.

In some examples, the specific disease or disease state is a cancer. A variety of cancers can be detected or identified. In some examples, the cancer is adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic disease, head and neck cancer, Hodgkin lymphoma, intestinal cancer, kidney cancer, leukemia, liver cancer, lung cancer, melanoma, mesothelioma, multiple myeloma, neuroendocrine tumors, non-Hodgkin lymphoma, oral cancer, ovarian cancer, pancreatic cancer, prostate cancer, sinus cancer, skin cancer, soft tissue sarcoma, spinal cancer, stomach cancer, testicular cancer, throat cancer, thyroid cancer, uterine cancer, vaginal cancer, or vulvar cancer. Thus, in examples, the magneto-plasmonic nanorods disclosed herein include an antibody (or portion thereof) for identifying and isolating exosomes as well as a labeled detectable molecule (such as a fluorescently labeled hairpin DNA that includes a sequence complementary to one or more miRNA associated with one or more cancers). In some examples, the miRNA includes one or more of hsa-let-7b-5p, hsa-miR-10b-5p, hsa-miR-125b-5p, hsa-miR-155-5p, hsa-miR-16-5p, hsa-miR-191-5p, hsa-miR-20a-5p, hsa-miR-214-3p, hsa-miR-21-5p, hsa-miR-222-3p, hsa-miR-106b-5p, hsa-miR-320a, hsa-miR-191-5p, hsa-miR-223-3p, hsa-miR-373-3p, hsa-miR-375, hsa-miR-21-5p, hsa-miR-21-5p, hsa-miR-122-5p, hsa-miR-21-5p, hsa-miR-425-5p, hsa-miR-141-3p, hsa-miR-27a-3p, hsa-miR-29a-3p, hsa-miR-130b-3p, hsa-miR-24-3p, hsa-miR-24-3p hsa-miR-486-5p, hsa-miR-20a-5p, or hsa-miR-100-5p (for example, using a serum sample) or hsa-miR-106b-5p, hsa-miR-21-5p, hsa-miR-21-5p, hsa-miR-155-5p, hsa-miR-21-5p hsa-miR-21-5p, hsa-miR-141-3p, hsa-miR-18a-5p, hsa-miR-29a-3p, hsa-miR-122-5p, hsa-miR-196a-5p, hsa-miR-155-5p hsa-miR-210-3p, hsa-miR-199a-3p, hsa-miR-15b-5p, hsa-miR-223-3pb, hsa-miR-222-3pb, hsa-miR-210-3p, hsa-miR-21-5p, hsa-miR-486-5pb, hsa-miR-451a, or hsa-miR-486-5p (for example, using a plasma sample).

In some embodiments, the methods can include measuring differential expression of one or more cancer-associated miRNAs compared with a control (for example, a subject, which does not have a cancer), thereby identifying a cancer in the subject. The methods may further include treating the subject for cancer. For example, the methods can include administering one or more of surgery, radiation therapy, chemotherapy, immunotherapy, targeted therapy, hormone therapy, stem cell transplant, and genetics-based precision medicine to the subject.

In additional examples, the methods can also be used to identify the level of cell differentiation (such as stem cell differentiation) in a population of cells. An exemplary method of characterizing stem cell differentiation is illustrated in FIG. 1C.

Methods of differentiating stem cells are known by persons skilled in the art (see, e.g., US Pub No. US20110086379A1; Int Pub No. WO2014062138A1; U.S. Pat No. 9,593,305B2, all of which are incorporated herein by reference in their entireties). In addition, biological components and associated molecules are known by persons skilled in the art to be associated with stem cell differentiation. In specific, non-limiting examples, the methods can be used to identify exosomes associated with one or more specific miRNA that are associated with stem cell differentiation. In specific, non-limiting examples, the methods include identifying and isolating exosomes using the nanorods herein and further measuring one or more specific miRNA expressed by exosomes, for example, with a fluorescently labeled hairpin DNA that includes a sequence complementary to the specific miRNA. In some embodiments, the methods include measuring differential expression of one or more miRNAs compared with a control stem cells that are not differentiated. Differentiated stem cells can be used in a variety of applications.

In some examples, the methods further include administering a stem cells treatment to a subject for a disease. Examples of diseases for which stem cell treatments are of use include diabetes, rheumatoid arthritis, Parkinson's disease, Alzheimer's disease, osteoarthritis, stroke and traumatic brain injury repair, learning disability due to congenital disorder, spinal cord injury repair, heart infarction, cancer, baldness, missing teeth, hearing damage, vision damage (such as corneal damage), amyotrophic lateral sclerosis, Crohn's disease, wound healing, male infertility, and female infertility. Examples of miRNA associated with differentiated stem cells include miRNA-124, miR-451a, miR-491-5p, miR-338-3p, miR-21-5p, miR-29b-3p, miR-451a, miR-335-5p, miR-338-3p, miR-21-5p, miR-17-5p, miR-126-5p, miR-126-3p, miR-145-5p, miR-335-5p, miR-145-5p, miR-222-3p, miR-203a, miR-145-5p, miR-9-5p, miR-100-5p, miR-27b-3p, miR-140-5p, miR-27a-3p, miR-125b-5p, miR-143-3p, miR-132-3pmiR-519a-3p, miR-26b-5p, miR-519d-3p, miR-519c-3p, miR-200c-3p, miR-181b-5p, miR-21-5p, miR-1, miR-222-3p, miR-221-3p, miR-103a-3p, miR-335-5p, miR-124-3p, miR-423-5p, miR-30c-2-3p, miR-18b-5p, miR-16-5p, let-7e-5p, miR-24-3p, miR-29b-3p, miR-144-3p, miR-633, miR-663a, miR-21-5p, miR-124-3p, miR-18a-5p, miR-26a-5p, miR-205-5p, miR-145-5p, miR-375, miR-19b-3p, and miR-19a-3p.

### EXAMPLES

The following examples are provided to illustrate certain particular features and/or embodiments. These examples should not be construed to limit the disclosure to the particular features or embodiments described.

### Example 1

### Materials and Methods

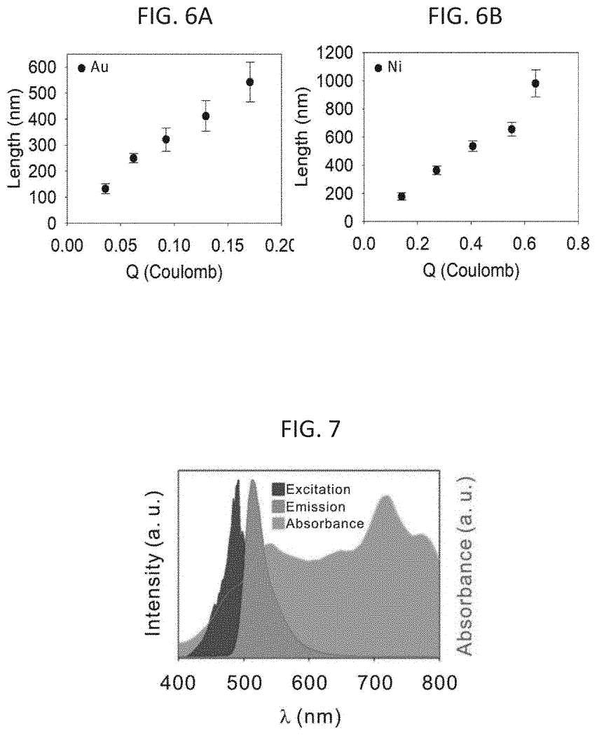

Generation of Magneto-Plasmonic Nanorods via Electrodeposition. The generation method for magnetic-gold nanorods was adapted from previous reports (Martin, C. R., Science 266:1961-1966, 1994; Lee et al., J. Phys. Chem. C 116:18388-18393, 2012; Banholzer et al., Nat. Protoc. 4:838-848, 2009). Briefly, a thin layer of silver (approximately, 300 nm) was evaporated on one side of an AAO template (Anodisk™ 13, Whatman®) by physical vapor deposition as a conducting layer. By providing electrical contact with aluminum foil in the Teflon cell with a Ag layer on one side of the AAO template, the AAO template served as a working electrode in an electrochemical setup. In addition, a Ag/AgCl and a Pt wire served as the reference and counter electrode to form a three-electrode configuration. To block the irregular branch part of the AAO nanopores, Ag was predeposited into AAO by a commercial plating solution at −0.95 V (vs Ag/AgCl). In order to synthesize the magneto-plasmonic nanorods, approximately 250 nm of both Ni and Au components were sequentially deposited by a commercial plating solution at −0.95 V (vs Ag/AgCl). The desired configuration and length of each block of nanorods were controlled by the type of plating solution selected and the depositing Coulomb value applied. Specifically, ca. 0.07 Coulomb was used for growth of a 250 nm block of Au, and ca. 0.20 Coulomb was used for the growth of a 250 nm block of Ni. The lengths of the Au and Ni components were measured from SEM images obtained by cross-deposition of Au and Ni on an AAO template with varying charges (Coulombs). The correlation between the Coulomb values applied for electrochemical growth of each component (Au and Ni) was characterized using FE-SEM (Sigma®, Zeiss, Germany). Twenty random components from each condition were used to measure the length of each component (FIGS. 6A and 6B). The Ag layer was etched with a 4:1:1 ratio mixture of methanol (CH₃OH), hydrogen peroxide (H₂O₂) (30% vol/vol), and ammonium hydroxide (NH₄OH) (28% in H₂O), respectively (Banholzer et al., Nat. Protoc. 4:838-848, 2009).

The AAO template was then completely removed by 3 M NaOH for 50 min (Lee et al., J. Phys. Chem. C 116:18388-18393, 2012). The resulting samples were rinsed with distilled water and visualized with an FE-SEM on an indium tin oxide-coated glass substrate.

Preparation of Multifunctional Magneto-Plasmonic Nanorods. Each component of the magneto-plasmonic NRs was functionalized with a FAM-tagged molecular beacon (MB) against miR-124 and antibody against CD63, respectively (Table 1). First, presynthesized magneto-plasmonic NRs were washed with phosphate-buffered saline (PBS) three times using centrifugation (centrifuge 5415R; Eppendorf®, Germany). To selectively functionalize the surface of the Au component only, magnetic-gold NRs were incubated with thiol group-functionalized MB (final concentration 10 μM; Table 1) and 1,4-dithiothreitol (C₄H₁₀O₂S₂) (final concentration 100 μM) for 8 h at 4° C. to promote covalent bonding between Au and thiol. After functionalizing the Au block, magnetic-gold NRs were washed with PBS three times and incubated with the anti-CD63 antibody (final concentration range from 10 μg/mL) to functionalize the Ni component as well. After an 8 h incubation period at 4° C., 3% bovine serum albumin (BSA) was added to the mixed solution to stabilize the multifunctional magneto-plasmonic NRs.

TABLE 1
Example sequences for a molecular
beacon with a fluorophore and genes
(positive, single mismatch, and
negative control) analyzed for
a cell-free condition.

| | Sequence | Size |
|---|---|---|
| Gene | (5′ to 3′) | (nt) |
| Molecular | 6-FAM/GCGCCATTCA | 19 |
| Beacon-19 | CCGCGGCGC/ | |
| | ThioMC3-D | |
| | (SEQ ID | |
| | NO: 1) | |
| Molecular | 6-FAM/GCGCCATTCACC | 25 |
| Beacon-25 | GCGTGCCTGGCGC/ | |
| | ThioMC3-D | |
| | (SEQ ID NO: 2) | |
| Molecular | 6-FAM/GCGCCATCATTCA | 31 |
| Beacon-31 | CCGCGTGCCTTTTGGCGC/ | |
| | ThioMC3-D | |
| | (SEQ ID NO: 3) | |
| Molecular | 6-FAM/GCGCCATTTTCA | 37 |
| Bcacon-37 | TTCACCGCGTGCCTTTT | |
| | TTTGGCGC/ | |
| | ThioMC3-D | |
| | (SEQ ID NO: 4) | |
| Molecular | 6-FA M/GCGCCATTT | 43 |
| Beacon-43 | TTTTCATTCACCGCGT | |
| | GCCTTTTTTTTTTGGC | |
| | GC/ThioMC3-D | |
| | (SEQ ID NO: 5) | |
| Molecular | 6-FAM/GCGCCATTTTT | 49 |
| Beacon-49 | TTTTTCATTCACCGCGT | |
| | GCCTTTTTTTTTTTTTG | |
| | GCGC/ThioMC3-D | |
| | (SEQ ID NO: 6) | |
| Molecular | 6-FAM/GCGCCAGCTAA | 25 |
| Bcacon-25 | CAATACACTGGCGC/ | |
| (miR-449-a) | ThioMC3-D | |
| | (SEQ ID NO: 7) | |
| Positive | TTAAGGCACGCGGTGAA | 22 |
| Control | TGCCA | |
| (miR-124) | (SEQ ID NO: 8) | |
| Single | TTAAGGCACGTGGTGAA | 22 |
| Mismatch | TGCCA | |
| | (SEQ ID NO: 9) | |
| Negative | TCACAACCTCCTAGAAAG | 24 |
| Control | AGTAGA | |
| (miR-67) | (SEQ ID NO: 10) | |

Fluorescence Measurements and Experimental Setup. Anti-CD63 antibody and FAM-tagged miR-124-targeting MB-modified multifunctional magneto-plasmonic NRs (20 mg/mL) were mixed with cell culture medium (1 mL), which was obtained during neuronal differentiation of human stem cells. After 30 min, magneto-plasmonic NRs were isolated under a magnetic field (BioMag®, Polyscience Inc.) and resuspended in a transparent 96-well plate with PBS with 0.1% Tween 20 (100 μL). The fluorescence spectra were recorded with an excitation of 490 nm and emission of 520 nm at 25° C. using a 96-well plate reader (Infinite® M200pro, TECAN Group, Ltd., Switzerland).

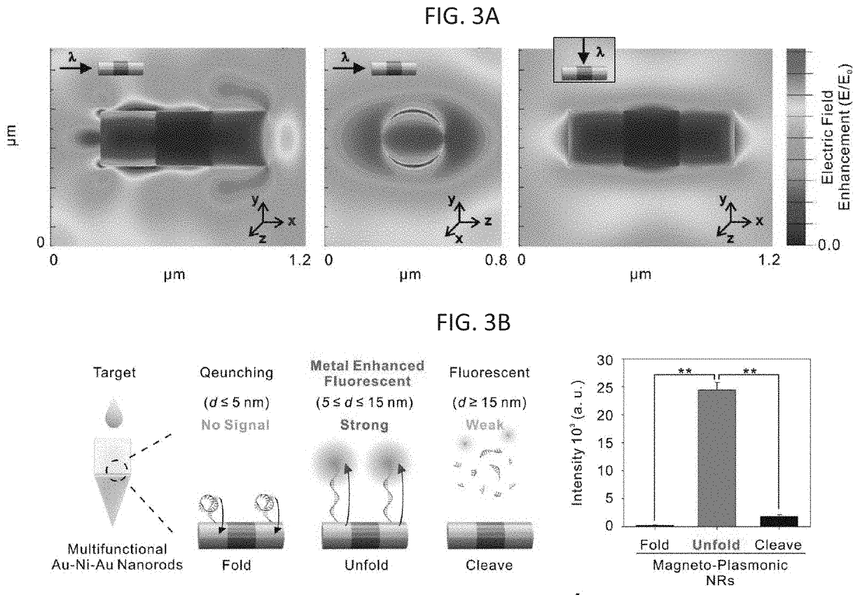

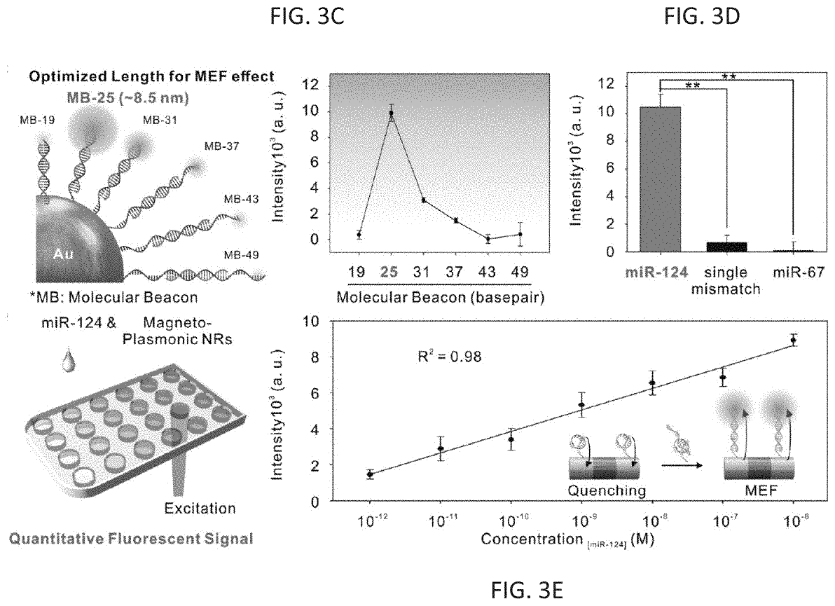

Finite-Difference Time-Domain Simulation. To study the metal-enhanced fluorescence surrounding the multicomponent magneto-plasmonic NRs, electromagnetic field enhancement surrounding the magneto-plasmonic NRs was simulated and calculated using the FDTD package provided by Lumerical. To match the fluorescence excitation wavelength, a plane wave light source model with a single wavelength of 490 nm was introduced. Magneto-plasmonic NRs with a diameter of 278 nm and heights of 248, 250, and 248 nm for each section were used to be consistent with the nanorods we synthesized. Re (index) and Im (index) were used from the materials library without further modifications. To simulate light from different directions, two models were calculated: one model with the light source vertical to the longitudinal axis of the NR and the other model with the light source horizontal to the longitudinal axis of the NR. In the vertical model, the light source was placed at 400 nm on top of the NR, while in the horizontal model the distance is kept at 500 nm. A mesh size of 4 nm was utilized for all simulations, and the media surrounding the nanorods were set as a vacuum. Monitors were set at different locations of NRs with fixed wavelengths of 490 nm. The electromagnetic field was calculated and plotted in the heat map of E/EO and summarized in FIG. 3A.

Cell Culture and Differentiation. The human induced pluripotent stem cell-derived neural stem cells were maintained in mixture of neural basal medium (Gibco™) and DMEM/F12 (Gibco™) (50:50 ratio) supplemented with 0.5% N2 (Gibco™), 0.5% B27, and 20 ng/mL FGF basic (Fibroblast growth factor-basic, PeproTech®), respectively. All cells were maintained at 37° C. in a humidified incubator with 5% CO₂. To differentiate cells, hiPSC-NSCs were seeded on Matrigel® (Life Technologies)-precoated plates (300 000 cells/well in a six-well plate) 24 h prior to experimentation. After 1 day of cultivation to promote cell attachment and spreading, the fresh hiPSC-NSC media without FGF basic (differentiation media) was treated to stop proliferation and induce neuronal differentiation. The medium was changed with fresh differentiation media every 3 to 4 days during the differentiation. For consistency, all experiments were carried out on cells between three passage differences. Human mesenchymal stem cells (American CryoStem®) were maintained in Alpha MEM (L-glutamine) supplemented with 10% SCM141 (PLTMax® human platelet lysate), 30 mg of heparin sodium salt, and 1% penicillin/streptomycin (Gibco™). The human cerebral microvascular endothelial cells were cultured in the human endothelial cell culture medium (Cell Applications, Inc.) supplemented with 5% fetal bovine serum (FBS), penicillin (100 units/mL), streptomycin (100 μg/mL), 1.4 μM hydrocortisone, 5 μg/mL ascorbic acid, 10 mM HEPES, and 1 ng/mL basic fibroblast growth factor (bFGF). The astrocytes were maintained in the astrocyte culture medium (Sciencell) with 10% FBS, penicillin (100 units/ml), and streptomycin (100 μg/mL). Human dermal fibroblasts were cultivated in fibroblast growth medium (Cell Applications, Inc.). For collection of the exosomes from each of the cell lines, 3×10⁶ cells were seeded in a six-well plate, and each media were collected after 3 days from the seeding. ReNcell® cells (human neural progenitor cell line) were obtained from Merck and maintained in DMEM/F12 (Gibco™) supplemented with 1% L-glutamine (200 nM, Invitrogen™), 2% B27, 20 ng/mL heparin (Sigma-Aldrich™), gentamycin (10 m/mL), 0.1% bFGF (Sigma-Aldrich™), and 0.1% EGF (epidermal growth factor, Sigma-Aldrich™), respectively. In the case of ReNcell® cell differentiation, a similar protocol was used with hiPSC-NSCs. ReNcell® cells were seeded on Matrigel®-precoated plates (2,000,000 cells/well for six-well plates) 24 h prior to experimentation. After 1 day of cultivation to promote cell attachment and spreading, the fresh ReNcell® media without bFGF basic (differentiation media) was treated to stop proliferation and induce neuronal differentiation. ReNcell® cells and astrocytes were cocultured in one dish, and neural differentiation was induced. Both were maintained in a mixture of DMEM/F12 (Gibco™) and astrocyte culture medium (1:1 ratio) with all supplements. Total seeding number of cells was 2.0×10⁶, which corresponds to 1:9/1:4/1:1/ 4:1/9:1 ratios for ReNcell® cells and astrocytes, respectively. ReNcell® cells and astrocytes were seeded on Matrigel®-precoated plates 24 h prior to experimentation. After 1 day of cultivation to promote cell attachment and spreading, the fresh mixture of cell media without bFGF basic was treated to stop proliferation and induce neuronal differentiation. The medium was changed with fresh differentiation media every 3 to 4 days during the differentiation. The culture media was collected 10 days after the induction of neural differentiation. Next, the media were centrifuged at 2000 rpm for 5 min to separate the cell debris. For consistency, all experiments were carried out on cells between passages 3 and 5.

Immunocytochemistry. To study the extent of neuronal differentiation, cells and explanted brain tissue were washed with PBS (pH 7.4) and fixed with 4% formaldehyde solution for 10 min at room temperature, followed by washing with PBS three times. Next, cells were permeabilized with 0.1% Triton™ X-100 in PBS for 10 min, and nonspecific binding was blocked with 5% normal goat serum (NGS) (eLife Technologies) in PBS for 1 h at room temperature. The primary rabbit antibody against Nestin (1:200 dilution, Invitrogen™) and primary mouse antibody against TuJ1 (1:200 dilution, Biolegend) were used for the cells. Moreover, the primary rabbit antibody against GFAP (1:200 dilution, Invitrogen™) and primary mouse antibody against TuJ1 (1:200 dilution, Biolegend) were used for the explanted brain tissues. Following the manufacturer's protocol, the fixed samples were incubated overnight at 4° C. in a solution of this antibody in PBS containing 1% BSA and 0.3% Triton™ X-100. After washing three times with PBS, the samples were incubated for 1 h at room temperature in a solution of anti-rabbit secondary antibody labeled AlexaFluor® 488 (1:100, Life Technologies), anti-mouse secondary antibody labeled with AlexaFluor® 647 (1:100, Life Technologies), and Hoechst (3 μg/ mL, Life Technologies) to stain nuclei in PBS containing 1% NGS and 0.3% Triton™ X-100. After washing three times, all the samples were imaged using a Nikon T2500 inverted fluorescence microscope.

Gene Expression Analysis. Gene expression level was analyzed by quantitative reverse transcription PCR from total RNA extracted from cells by a TRIzol® reagent (Invitrogen™, MA, USA). The total RNA (1 μg) was reverse transcribed to cDNA using the SuperScript III™ first-strand synthesis system (Invitrogen™, MA, USA) following the manufacturer's protocol. Subsequently, quantitative PCR was performed on a StepOnePlus™ real-time PCR system (Applied Biosystems™, MA, USA) using a SYBR™ Green PCR master mix (Applied Biosystems™) with the gene-specific primers, listed in Table 2. The standard cycling conditions were used for all PCR reactions with a melting temperature of 60° C. All the measurements were run in triplicate. The gene expression level was reported relative to the endogenous control gene, GAPDH.

TABLE 2
Example primer sequence
for analyzed genes.

| Gene | Forward Primer | Reverse Primer |
|---|---|---|
| GAPDH | CCGCATCTTCT | GCCCAATACGAC |
| | TTTGCGTCG | CAAATCCGT |
| | (SEQ ID NO: 11) | (SEQ ID NO: 12) |
| Nestin | CAACAGCGACG | GCCTCTACGCTCTC |
| | GAGGTCTC | TTCTTTGA |
| | (SEQ ID NO: 13) | (SEQ ID NO: 14) |
| TuJ1 | GGACCCTGTGAG | CAGCTCCGAC |
| | TAGCCAGTA | AGATCCAGT |
| | (SEQ ID NO: 15) | (SEQ ID NO: 16) |

Quantification of Intracellular miRNA Expression. Intracellular miRNA expression was quantified throughout differentiation (21 days). At each time point, the total RNA, including miRNA, was extracted from cultured cells using the miRNeasy micro kit (Qiagen®, MD, USA) following the manufacturer's protocol. The first-strand cDNA was synthesized from total RNA (50 ng) using a universal reverse transcription reaction system offered in the miRCURY® LNA® RT kit (Qiagen®). The as-synthesized cDNA template was diluted 60 times prior to real-time PCR amplification on a StepOnePlus™ real-time PCR System (Applied Biosystems™, MA). Each real-time PCR was carried out in a miRCURY® LNA® miRNA PCR system (Qiagen®) in a 10 μL reaction with 3 μL of cDNA, 5 μL of 2×miRCURY® SYBR™ Green master mix, and the miRNA-specific PCR assays (primers): hsa-miR-124-5p as miR-124-specific primers and hsa-miR-103a-3p as the endogenous control. The two-step cycling conditions were performed as follows: Initiation activation at 95° C. for 2 min, 40 cycles of denaturation at 95° C. for 10 s, and combined annealing/extension at 56 ° C. for 60 s. The resulting C_{T} values were normalized and reported in fold changes relative to the endogenous control (miR-103a-3p). All measurements were repeated three times.

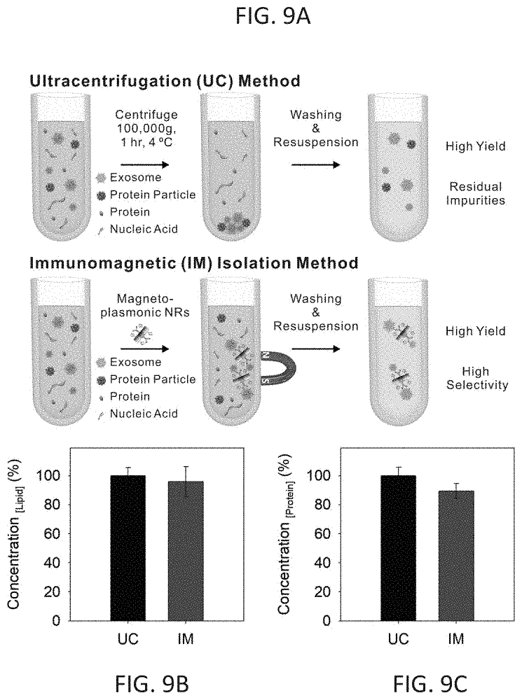

Confirmation of Extraction and Concentration of Exosomes via Magneto-Plasmonic Nanorods Based on HRP-TMB Reaction. Anti-CD63- and miR-124-targeting MB-functionalized magneto-plasmonic NRs (20 mg/mL) were mixed with cell culture medium (1 mL), which was obtained during neuronal differentiation of hiPSC-NSCs. After 30 min, NRs were isolated under a magnetic field and resuspended with the anti-CD9-HRP antibody (Santa Cruz Biotechnology, Inc.) in PBS (final concentration 10 m/mL). After 30 min, multifunctional magneto-plasmonic NRs were repeatedly isolated under a magnetic field and resuspended with PBS three times for separation of unbound anti-CD9-HRP. After the washing step, TMB (MP Biomedicals, Inc.) was added to the multifunctional magneto-plasmonic NR solution for 30 min. The UV/vis absorbance value was obtained at 650 nm using a 96-well plate reader (Infinite® M200pro, TECAN Group, Ltd., Switzerland). In addition, to validate the exosome isolation efficiency and selectivity of our system, the immunomagnetic isolation method exemplified herein was compared to the ultracentrifugation method (centrifugation with 100,000×g for 1 h at 4° C., Optima XE-90 with SW41 Ti rotor; Beckman Coulter, USA) (FIG. 9A). The overall lipid concentrations were measured (lipid assay kit, Abcam, UK) from the collected exosomes after the isolation procedures. As shown, the isolation efficiency of the immunomagnetic (IM) isolation method (95.8±10.3%) was comparable to the ultracentrifugation (UC) method at the condition of 100 mg/mL of multifunctional magneto-plasmonic NRs for 1 mL of biological fluid (FIG. 9B). The bicinchoninic acid (BCA) assay was also performed to measure the overall protein concentration from the collected exosomes after the isolation procedures to validate the specificity of exosome isolation methods (UC and IM) (FIG. 9C). Compared to lipid quantification, protein quantification (BCA assay) (89.3±5.1%) showed a large signal difference between UC and IM. The overall exosome isolation efficiency of the example platform disclosed herein was comparable the ultracentrifugation isolation method. In addition, example platform disclosed herein has the advantage of improved selectivity from the attached antibody specific to exosomes on the surface of multifunctional magneto-plasmonic NRs.

Nondestructive Characterization of Neural Cell Distribution in the Explanted Brain Tissues. CD1 postnatal day 4 (P4) mice brain tissue was dissected and maintained in a mixture of neural basal medium (Gibco™) and DMEM/F12 (Gibco™) (50:50 ratio) supplemented with 0.5% N₂ (Gibco™) and 0.5% B27, respectively. Brain tissue was maintained at 37° C. in a humidified incubator with 5% CO₂. After 1 day of cultivation, nonadhered tissue was removed, and the fresh medium was introduced. The medium was changed with fresh differentiation media every 3 to 4 days during differentiation. For the collection of the exosomes, media were collected after 3 days from the explant. The cultured media were then centrifuged at 2000 rpm for 5 min to separate the cell debris.

### Example 2

### Generation and Characterization of Multicomponent Magneto-Plasmonic Nanorods

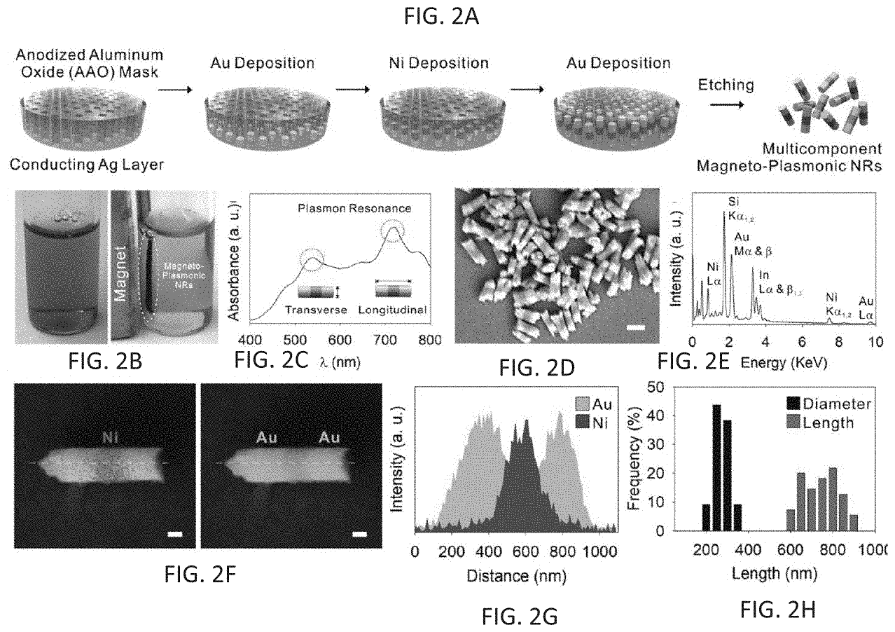

To develop the magneto-plasmonic NR-based efficient exosome isolation and exosomal miRNA detection method, homogeneous multicomponent magneto-plasmonic NRs were synthesized through potentio-static electrochemical deposition utilizing AAO (pore size 0.2 μm) as a template (FIG. 2A). The diameter and the length of the multicomponent magneto-plasmonic NRs could be independently tuned by varying the pore size of AAO templates and the applied total electrical charge, respectively (FIGS. 6A-6B). Specifically, adjusting the diameter and the length of the multicomponent magneto-plasmonic NRs facilitated tuning of the aspect ratio as well as the property of the localized surface plasmon resonance (LSPR). In the—multicomponent magneto-plasmonic NR system, the Au-Ni-Au configuration was used to protect the Ni component during the etching process (Lee et al., J. Phys. Chem. C 116:18388-18393, 2012). The ferromagnetic Ni component facilitated magnetic isolation of the multicomponent magneto-plasmonic NR from solution (FIG. 2B), and the LSPR-active Au component (at 540 and 720 nm) facilitated fluorescence signal amplification (FIGS. 2C and 7). As demonstrated, the multicomponent magneto-plasmonic NRs are a hybrid nanomaterial having orthogonal magnetic and optical properties. The distinct separation of the magnetic and plasmonic components is crucial for the orthogonal conjugation chemistry of the synthesized NRs for efficient exosome capture and selective exosomal miRNA detection. To this end, the structure and distinct regions of the multicomponent magneto-plasmonic NRs were verified through field emission scanning electron microscopy (FE-SEM; FIG. 2D). Through energy dispersive X-ray (EDX) analysis, the multicomponent magneto-plasmonic NRs were shown to have a bimetallic multicomponent structure with definite segregation of the Au and Ni elements (FIGS. 2E-2G). In detail, distinct X-ray emission peaks of Mα and β of Au were observed at around 2.1 keV, Lα of Au at around 9.7 keV, Lα of Ni at around 0.8 keV, and Kα1,2 of Ni at around 7.5 keV, which clearly demonstrates the presence of both Au and Ni components on the multicomponent magneto-plasmonic NRs. The presence of Kα1,2 of Si (silicon) at around 1.7 keV and Lα, β1,2, and γ1 of In (indium) at around 3.3, 3.5, 3.7, and 3.9 keV was due to the sample holder (indium tin oxide-coated glass substrate) for FE-SEM imaging. The magneto-plasmonic NRs were designed to be larger than exosomes (30-100 nm) in order to extract exosomes and concentrate them. For this purpose, the ferromagnetic Ni component was designed to be 250 nm in length and diameter. From the SEM analysis, the multicomponent magneto-plasmonic NRs had a homogeneous size distribution and were found to be 267.35±23.78 nm in diameter and 745.31±89.09 nm in length with an aspect (ratio of length/width) ratio of 3 (FIG. 2H).

Surface Functionalization of Multicomponent Magneto-Plasmonic Nanorods for miRNA Detection. The selective and sensitive detection of stem cell neurogenesis markers in a nondestructive manner is crucial for characterizing cell identities before effective clinical applications. For nondestructive characterization of neural differentiation, on the two ends of the multifunctional magneto-plasmonic NR, an miR-124 (miRNA-124)-specific MB (molecular beacon) was attached on the surface of the plasmonic Au blocks (Table 1). miR-124 was used as a specific biomarker for stem cell neurogenesis due to its low expression levels in NSCs and high expression level in differentiated neurons (Papagiannakopoulos et al., Cell Stem Cell 4:375-376, 2009). For the sensitive detection of neurogenesis, the distinct LSPR property of gold nanostructures was examined, in which the gold nanostructures can both quench and enhance the fluorescence signal from the fluorophore attached. Accordingly, the MB (length of complementary strands approximately 5 to 15 nm) were designed to control the leading phenomena between quenching and enhancement of fluorescence signal (Aslan et al., Curr. Opin. Biotechnol. 16:55-62, 2005; and Kuhn et al., Phys. Rev. Lett. 97:017402, 2006). Moreover, 5(6)-carboxyfluorescein (FAM) was selected as the fluorophore of the MB, as its emission band (520 nm) matches up with the transverse plasmon absorption of the magneto-plasmonic NRs (540 nm) to generate MEF effects (FIG. 7; Chen et al., Nano Lett. 7:690-696, 2007; Li et al., Analyst 140:386-406, 2015). To maximize the MEF effect and provide a theoretical explanation, finite-difference time-domain (FDTD) simulation was performed using the electromagnetic (EM) field surrounding the multicomponent magneto-plasmonic NRs to investigate the fluorescence signal amplification of FAM (FIGS. 3A-3E). Based on the parameters derived from physical measurements obtained through SEM analysis, the structures of the multicomponent NRs were defined in the FDTD simulation to ensure that the simulation results precisely represented the experimental conditions (FIG. 2H). The local electric field enhancement [(|E|/E₀|)] was defined as the ratio between the near-field (|E|) intensity and the incident field (|E₀|) intensity, which determined the extent of excitation enhancement due to MEF. The calculated EM field enhancement distribution images indicated that, when multicomponent magnetic-gold NRs were exposed to incident light in either direction, longitudinal (FIG. 3A, left and middle) or transverse (FIG. 3A, right), the local electric field enhancement significantly increased around the Au components.

To verify the ability of the MB-functionalized magneto-plasmonic NRs to both quench and enhance fluorescence signal during the inactive and active states, the fluorescence signal readings from three different conditions were quantified (folded MB, unfolded MB, and cleaved MB; FIG. 3B). By varying the temperatures, no observable fluorescence signal was obtained from the folded MB on the magneto-plasmonic NR sample due to fluorescence quenching at room temperature. At elevated temperature (65° C.), the MB strand unfolded, and a clear and strong fluorescence signal at 520 nm was observed. The cleavage of MB from the magneto-plasmonic NR surface using exonuclease I (Exol), on the other hand, showed 15-fold less fluorescence signal compared to the unfolded state. These results clearly demonstrate the robustness of the MEF effect obtained by the magneto-plasmonic NR-based exosomal miRNA detection method. The enhancement of the fluorescence signal was examined by testing different lengths of MBs from 19 to 49 bps (6.5 to 16.7 nm in length). Due to the quenching and MEF effect, MB-25 (25 bps, 8.5 nm) showed the strongest fluorescence signal (FIG. 3C), while the fluorescence signal was quenched for the shorter MB and significantly decreased for the longer MBs.

Next, the specificity of the multifunctional magneto-plasmonic NR-based detection method was validated by examining the synthetic miR-124-targeting MB. Using a solution-based assay, a strong fluorescence signal was observed from hybridization between the synthesized miR-124-targeting MB and complementary DNA sequences (positive control). However, when miR-124-targeting MB interacted with single-base-pair-mismatched DNA and miRNA-67 (miR-67) DNA sequence (negative control), little to no fluorescence signals were observed (FIG. 3D and Table 1). In addition, the MB-functionalized magneto-plasmonic NR-based miRNA detection method showed good linearity (R²=0.98) at different concentrations (ranging from 1 pM to 1 μM) of the complementary DNA sequence of miR-124 (FIG. 3E), is consistent with miRNA assays in the literature (Qiu et al., ACS Nano 9:8449-8457, 2015; Graybill et al., Anal. Chem. 88:431-450, 2016; Mandiannasser et al., Biosens. Bioelectron. 107:123-144, 2018). Thus, the multicomponent magneto-plasmonic NRs based on the miRNA detection method is highly selective and sensitive and can quantify the concentrations of miR-124 in exosomes secreted by cells.

### Example 3

### Nondestructive, Selective, and Sensitive Characterization of Neurogenesis of hiPSC-NSCs through Immuno-Magnetically Concentrated Exosomal miRNA Detection

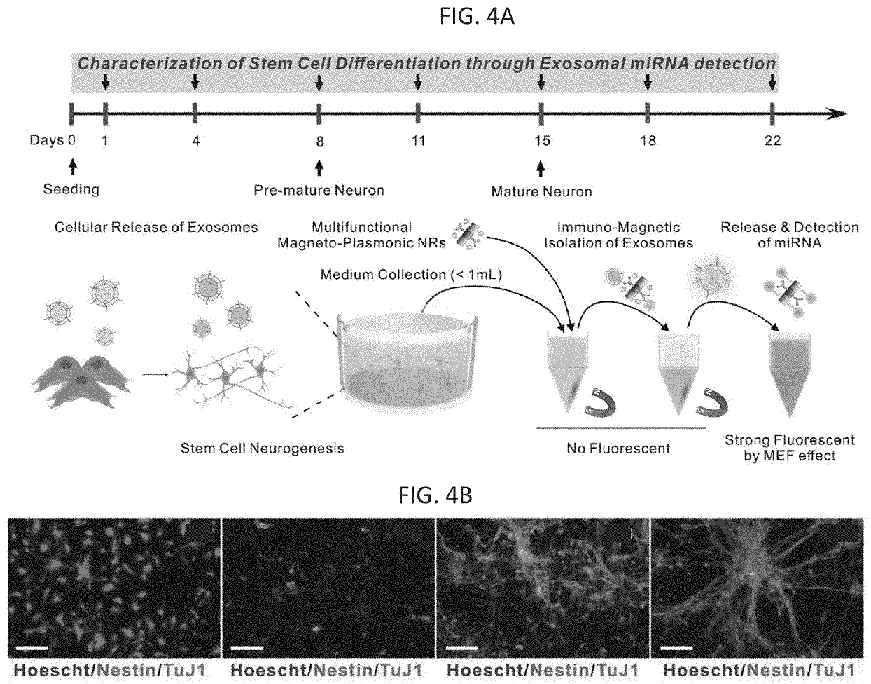

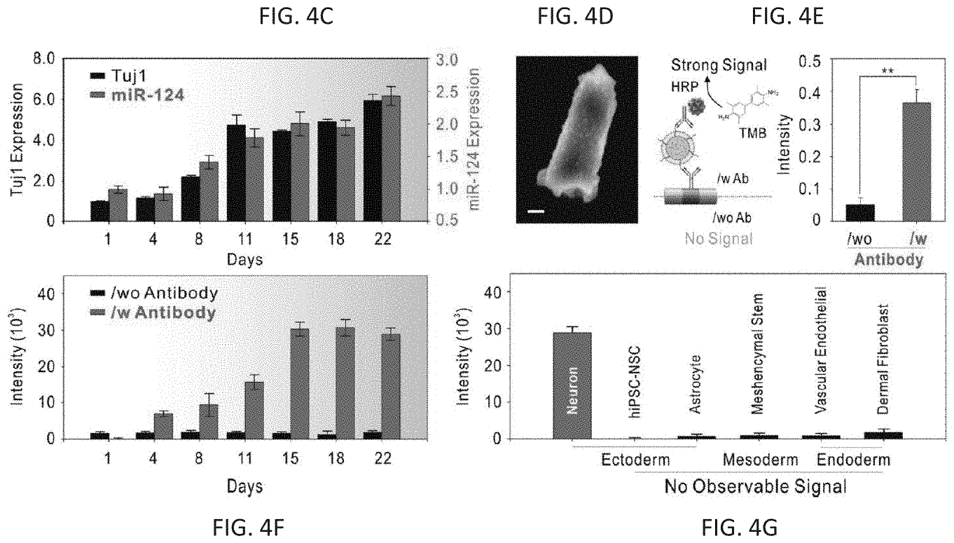

After confirming the selectivity and sensitivity of the magneto-plasmonic NRs miRNA detection platform, the nondestructive detection technique was applied to characterize neuronal differentiation of hiPSC-NSCs (FIG. 4A). For this purpose, the neuronal differentiation of hiPSC-NSCs was confirmed through immuno-cytochemistry staining of Nestin and neuron-specific class III β-tubulin (TuJ1), the representative markers of neural stem cells (NSCs) and differentiated neurons, respectively (Gage et al., Proc. Natl. Acad. Sci. U.S.A. 92:11879-11883, 1995; and Cheng et al., Cell Res.

24:665-679, 2014). As shown in FIG. 4B, TuJ1 expression (right middle, day 8 and right, day 22) was clearly observed in cells that had undergone neuronal differentiation, while the undifferentiated hiPSC-NSCs cells (left, day 1 and left middle, day 4) showed Nestin expression only without any significant TuJ1 expression. Additionally, reverse transcription polymerase chain reaction (RT-PCR) was performed over the differentiation period (3 weeks), and the expression trend of miR-124 was similar to Tujl mRNA expression. The expression level of miR-124 showed a time-dependent increase and saturation as the neuronal differentiation proceeded (FIG. 4C). Based on the well-correlated expressions of miR-124 with neuronal marker TuJ1, monitoring miR-124 expression is a reliable method for characterizing neuronal differentiation of hiPSC-NSCs.

To effectively capture and isolate exosomes, anti-CD63 IgG antibody was attached on the surface of the ferromagnetic Ni block of the magneto-plasmonic NRs for the immunomagnetic isolation and concentration of the targeted exosomes. Cell culture medium was collected throughout the neuronal differentiation period (3 weeks) for hiPSC-NSCs and immuno-magnetically isolate and concentrate exosomes to analyze exosomal miR-124 expression in a nondestructive, selective, and sensitive manner (FIG. 4A). The capture and concentration of exosomes by the multifunctional magneto-plasmonic NRs were verified through two independent methods: scanning electron microscope (SEM) analysis and a sandwich assay based on the 3,3′,5,5′-tetramethylbenzidine (TMB) reaction (Thery et al., Nat. Rev. Immunol. 9:581-593, 2009). The SEM analysis confirmed that the surface of the ferromagnetic Ni block region of the magneto-plasmonic NRs (functionalized with exosome-capturing anti-CD63 antibodies) was densely covered with cell-derived exosomes, demonstrating the efficient capture and isolation properties (FIG. 4D). Additionally, the TMB sandwich assay also showed a clear absorbance signal, while no observable signal was obtained from magneto-plasmonic NRs without anti-CD63 antibody functionalization (FIG. 4E).

After immunomagnetic isolation of exosomes, concentrated exosomes were lysed to release the encapsulated components, including miRNAs. As shown in FIG. 4F, no observable fluorescence signal was registered by the undifferentiated hiPSC-NSCs (day 1). In contrast, a distinct fluorescence signal became prominent starting from the premature (day 4) time point and saturating at the point of mature (day 15) neuron formation (FIG. 4F). Furthermore, the results collected from the nondestructive exosomal miRNA detection method were consistent with the results from the cell-lysing traditional RT-PCR technique for characterizing neuronal differentiation (FIG. 4C). In addition, due to the specificity of the miR-124 MB design, the multifunctional magneto-plasmonic NRs showed the ability to selectively and sensitively distinguish neurons from other types of ectoderm cells, including hiPSC-NSCs and astrocytes, as well as endoderm and mesoderm cells (FIG. 4G).

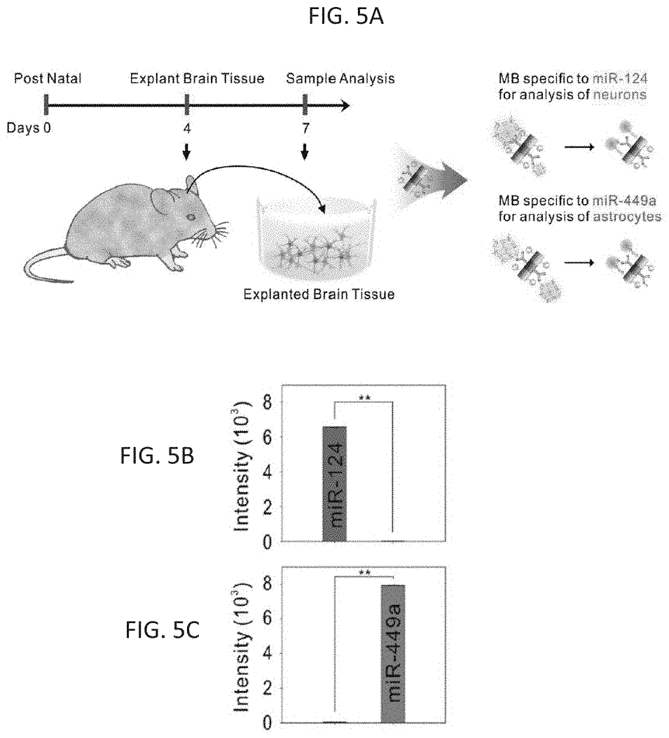

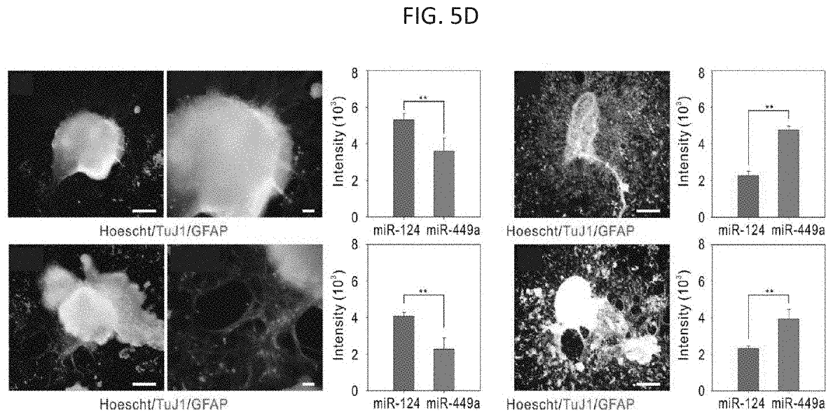

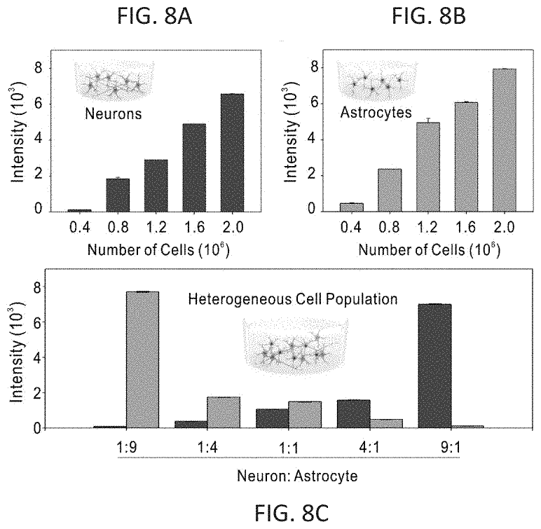

With the encouraging results from the above studies, the magneto-plasmonic NR was applied in a more complex, heterogeneous system, such as biological tissue. Because the supporting cells of the brain are predominantly astrocytes, an additional miRNA-449a (miR-449a) MB was designed to specifically target astrocytes (Table 1; Jovicic et al., J. Neurosci. 33:5127-5137, 2013; Rao et al., J. Neuropathol. Exp. Neurol. 75:156-166, 2016). In this proof-of-concept demonstration, the distribution of cell types in brain tissue was characterized (FIG. 5A). As above, the correlation between cell-type-specific exosomal miRNAs in neurons and astrocytes were examined separately using miR-124- and miR-449a-specific MB-functionalized magneto-plasmonic NRs (FIGS. 5B-5C). Neurons showed a fluorescent signal from miR-124-specific magneto-plasmonic NRs only and astrocytes showed a fluorescent signal with miR-449a-specific magnetic-gold NRs only. The fluorescent signals obtained from neuron- and astrocyte-derived exosomes positively correlated with the number of cells in the cell culture conditions (FIGS. 8A-8B). The ratiometric fluorescence signals collected from cocultured neurons and astrocytes at various ratios (FIG. 8C) show that the non-destructive, selective, and sensitive multifunctional magneto-plasmonic NR-based detection method is a reliable and quantitative exosomal miRNA detection method.

Due to the modular, interchangeable targeting moieties (e.g., target-specific MBs and antibody) and the nondestructive, selective, and sensitive neurogenesis characterization of the magneto-plasmonic NRs, different cell populations of mouse brain tissue explants were examined (FIG. 5A). In this ex vivo rodent model, brain tissues were dissected from a mouse (postnatal day 4) and cultured on Matrigel®-coated plates. During this step, brain tissue sections from different regions of the brain, such as the cerebral cortex, cerebellum, and the remainder of the brain, were adhered on Matrigel®-coated plates. After 3 days of cultivation, the cell culture medium was collected for analysis, and the tissues were fixed with formaldehyde solution. The distribution of neurons and astrocytes was characterized through immunocytochemistry staining of the neuron-specific marker (TuJ1) and glial fibrillary acidic protein (GFAP), which are representative markers of neurons and astrocytes, respectively. The populations of neurons and astrocytes varied from each brain tissue sample following the glia-to-neuron ratios reported for rodents depending on the sections of the brain (such as cerebral cortex, cerebellum, and the remainder of the brain; Herculano-Houzel, S., Glia 62:1377-1391, 2014). Using the magneto-plasmonic NRs-based exosomal miRNA detection method, correlative fluorescent signal ratios were demonstrated for each brain tissue sample with immunostaining results (FIG. 5D). These results show that the magneto-plasmonic NRs can be used to verify the distribution ratio of neurons and astrocytes in real tissues in a nondestructive, selective, and sensitive manner.

In view of the many possible embodiments to which the principles of the disclosed subject matter may be applied, it should be recognized that the illustrated embodiments are only examples and should not be taken as limiting the scope of the invention. Rather, the scope of the invention is defined by the following claims.

We claim:

1. A nanorod comprising a cylindrical body comprising a center portion and two flanking end portions, wherein:
the center portion comprises a magnetic composition coupled to at least one immunoactive macromolecule; and
the flanking end portions comprise a plasmonic composition coupled to at least one detectable molecule.

2. The nanorod of claim 1, wherein the magnetic composition comprises one or more of nickel, iron, or cobalt.

3. The nanorod of claim 1, wherein the plasmonic composition comprises one or more of gold, silver, or platinum.

4. The nanorod of claim 1, wherein the immunoactive macromolecule is an antibody or an antigen binding fragment thereof.

5. The nanorod of claim 1, wherein the detectable molecule is a detectably labeled peptide or a detectably labeled nucleic acid.

6. The nanorod of claim 5, wherein the detectably labeled peptide or nucleic acid is labeled with a fluorophore and a quencher.

7. The nanorod of claim 6, wherein the detectable molecule is a molecular beacon.

8. The nanorod of claim 1, wherein the detectable molecule specifically binds at least one target molecule associated with a specific vesicle.

9. The nanorod of claim 8, wherein the specific vesicle is an exosome.

10. The nanorod of claim 9, wherein the target molecule comprises an miRNA.

11. A kit comprising the nanorod of claim 1 and instructions for use.

12. A method of isolating exosomes from a sample, comprising:
contacting a sample with the nanorod of claim 1 under conditions sufficient for the nanorod to bind to the exosomes, thereby forming a nanorod-exosome complex;
isolating the nanorod-exosome complex from the sample; and
purifying exosomes from the nanorod.

13. The method of claim 12, wherein the sample comprises a biological sample from a subject or culture media from a population of cells or tissue.

14. The method of claim 12, wherein isolating the nanorod-exosome complex comprises contacting the complex with a magnetic field source.

15. A method of detecting a nucleic acid of interest in a sample, comprising:
contacting the nanorod of claim 1 with a sample comprising nucleic acids under conditions sufficient for the nanorod to specifically bind to the nucleic acid of interest, thereby forming a nanorod-nucleic acid complex, wherein the detectable molecule is a detectably labeled nucleic acid complementary to the nucleic acid of interest;
isolating the nanorod-nucleic acid complex from the sample;
measuring a signal from the detectably labeled nucleic acid; and
detecting the nucleic acid based on the signal from the detectably labeled nucleic acid.

16. The method of claim 15, wherein the sample comprises a biological sample from a subject or culture media from a population of cells or tissue.

17. The method of claim 15, wherein isolating the complex comprises contacting the complex with a magnetic field source.

18. The method of claim 15, wherein the labeled nucleic acid is a fluorescently labeled nucleic acid, and measuring the signal from the labeled nucleic acid, comprises:
applying an excitation wavelength; and
detecting an emission wavelength.

19. A method of treating a disease or disorder in a subject, comprising:
contacting the nanorod of claim 1 with a biological sample from a subject or culture media from a population of cells or tissue under conditions sufficient for the nanorod to specifically bind to a target associated with the disease or disorder, thereby forming a nanorod-target complex;
isolating the nanorod-target complex;
measuring a signal from the detectable molecule coupled to the nanorod;
identifying the disease or disorder or identifying therapeutically effective stem cells based on the signal from the detectable molecule; and
administering treatment to the subject.

20. The method of claim 19, wherein isolating the complex comprises contacting the complex with a magnetic field source.
